(19) United States
(12) Patent Application Publication
(10) Pub. No.: US 2025/0201336 A1
(43) Pub. Date: Jun. 19, 2025

(54) DIRECTED EVOLUTION OF MOLECULES BY ITERATIVE EXPERIMENTATION AND MACHINE LEARNING

(71) Applicant: Anagenex, Inc., Lexington, MA (US)

(72) Inventors: Svetlana BELYANSKAYA, Newton, MA (US); Polina BINDER, Tucson, AZ (US); George Joseph FRANKLIN, Peabody, MA (US); LaShadric Cederious GRADY, Lowell, MA (US); Meghan F. LAWLER, Tewksbury, MA (US); Henri PALACCI, Weston, CT (US); Nicolas TILMANS, San Francisco, CA (US); Sumudu Pamoda LEELANANDA, Menlo Park, CA (US)

(21) Appl. No.: 18/847,116

(22) PCT Filed: Mar. 15, 2023
(86) PCT No.: PCT/US2023/064354

§ 371 Date: Sep. 13, 2024

Related U.S. Application Data
(60) Provisional application No. 63/320,137, filed on Mar. 15, 2022.
(60) Provisional application No. 63/320,143, filed on Mar. 15, 2022.

(51) Int. Cl. G16B 15/30; G06N 20/00; G16B 5/20; G16B 35/20; G16B 40/20

(52) U.S. Cl. CPC G16B 15/30; G06N 20/00; G16B 5/20; G16B 35/20; G16B 40/20

(57) ABSTRACT

Described herein are platforms, systems, media, and methods for a machine learning-driven iterative drug discovery process. Some aspects comprise: receiving a first input data set comprising first binding interaction information between a target molecule and a library of compounds; processing the first input data set using a machine learning module to generate a model representation of binding interactions, wherein the representation is configured to predict binding between the target molecule and an input compound; determining an updated library of compounds using the model representation of binding interactions, wherein the updated library of compounds comprises one or more new compounds predicted to bind the target molecule; receiving a second input data set comprising second binding interaction information between the target molecule and the updated library of compounds; and processing the second input data set using the machine learning module to update the model representation of binding interactions, wherein the predictive accuracy of the updated model representation is improved.

### CROSS-REFERENCE TO RELATED APPLICATIONS

This application claims priority to U.S. provisional application 63/320,137, filed Mar. 15, 2022, and U.S. provisional application 63/320,143, filed Mar. 15, 2022, both of which are incorporated by reference herein in their entireties.

### BACKGROUND

Drug discovery is an expensive and time-consuming process in which candidate small molecule compounds, which can number in the millions, are screened for potential medicinal applications. Candidates can be identified through a number of approaches, including high-throughput screening (HTS), in which compounds are tested individually en masse with a biochemical assay, as well as in silico computational screening that evaluates target binding by modeling and/or calculating the molecular interaction. HTS is practically limited to a maximum of 2-3 million compounds, but is usually run with fewer. Meanwhile, computational screening requires a priori knowledge of both the crystal structure of the target and information where compounds should be binding on the target, and often does not accurately account for how a compound behaves in the real world.

### SUMMARY

Disclosed herein are platforms, systems, and methods for improved drug discovery with DNA encoded chemical libraries through directed molecular evolution utilizing an iterative machine learning process. Accordingly, clinically useful small molecules can be identified for a target and then rapidly optimized through the iterative process until the final candidate molecules are ready for clinical testing. Advantages of the present disclosure include the ability to identify molecules for a wide variety of target classes, find molecules for targets that others struggle with (i.e., undruggable), rapidly optimize compounds to in-vivo confirmation of an effect, and efficiently bringing multiple compounds to a clinic-ready stage. Instead of optimizing compounds by iterating slowly with a few dozen compounds a month, the present disclosure enables the creation of large datasets at each phase of the drug discovery cycle. By working with large, experimentally analyzed datasets (as opposed to in silico), multiparameter optimization can be efficiently carried out for complex multi-condition tasks like ADME/Tox (e.g., in a single experiment with a wide range of conditions).

The advantages of the present disclosure combine DNA encoded libraries (DELs) with machine learning to provide an improved screening process. The use of DELs provides advantages over high-throughput screening (HTS) and in silico computational approaches. For example, DELs allow 1,000 times more compounds to be processed than competing approaches (billions of compounds versus, at best, millions of compounds for HTS and traditional screening). DELs can be efficiently processed in 10-100× more parallel mini-experiments (“conditions”) than competing approaches, whereas HTS usually tests 1-2 conditions at a time due to the intense resource requirements for setting up each additional condition. In some cases, every combination of available building blocks is made in a DEL, allowing an exhaustive search for compounds, whereas HTS is heavily biased toward what has worked in the past, limiting the search space. For example, DELs do not require a crystal structure or a specific binding mode of action (unlike in silico virtual/computational screening). Instead, DELs can detect compounds that bind anywhere on the protein such as allosteric binders, cryptic binding pockets, and compounds that could be good foundations for bispecific molecules (e.g., PROTACs and other proximity-inducers such as molecular glues).

As a result, DELs can be applied to identify compounds that bind completely novel targets, currently dubbed “undruggable” because HTS does not cast a wide enough net to find these targets. Thus, the DEL approach generates rich, dense datasets full of internal controls, which is well suited to for machine learning (ML), while having lower cost and less time to screen than a traditional HTS.

The instant platforms, systems, and methods provide further advantages by integrating machine learning to generate ML-designed DELs in contrast to conventional HTS and DEL approaches that typically follow up on a few dozen compounds at a time.

In one aspect, provided herein is a method comprising: (a) a computer implemented method comprising: (i) receiving a first data set comprising: a first compound descriptor for each compound of a first library of compounds, and a compound fitness score for each compound of the first library of compounds; (ii) training a prediction model on the first data set; (iii) inputting into the model a second data set comprising a second compound descriptor for each compound of a second library of compounds; and (iv) generating from the prediction model a compound fitness score for each compound of the second library of compounds utilizing at least one or more compound descriptors of the first library of compounds and/or one or more compound descriptors of the second library of compounds, and (b) selecting a third library of compounds according to information comprising one or more compound fitness scores of the second library of compounds and/or one or more compound fitness scores of the first library of compounds. In some embodiments, the third library of compounds comprises: (i) a compound from the second library of compounds, (ii) a compound from the first library of compounds, (iii) a compound comprising two or more compounds from the second library of compounds, (iv) a compound comprising two or more compounds from the first library of compounds, (v) a compound comprising a compound from the second library of compounds and a compound from the first library of compounds, (vi) a compound not present in the first library of compounds or the second library of compounds, (vii) a compound comprising a compound from the second library of compounds and a compound not present in the first library of compounds or the second library of compounds, (viii) a compound comprising a compound from the second library of compounds and a compound not present in the first library or compounds or the second library of compounds, or (ix) a combination of two or more of (i) to (viii). In some embodiments, the first library a first DNA-encoded library (DEL) and/or the second library is a second DNA-encoded library. In some embodiments, step (b) is part of the computer implemented method. In some embodiments, wherein step (b) is not part of the computer implemented method. In some embodiments, step (b) comprises a first sub-step that is part of the computer implemented method and a second sub-step that is not part of the computer implemented method, wherein the first step and the second step are performed sequentially, and the first sub-step is performed first or the first sub-step is performed second.

In some embodiments, the information further comprises an assessment score (sometimes referred to as an external fitness score) of a compound of the second library of compounds and/or an assessment score (sometimes referred to as an external fitness score) of a compound of the first library of compounds. In some embodiments, the assessment score of the compound of the second library of compounds is a second fitness score generated independently from the compound fitness score generated from the computer implemented method. In some embodiments, the assessment score of the compound of the first library of compounds is a first fitness score that is different from the compound fitness score for the compound of the first library of compounds.

In some embodiments, one or more compounds of the first library is a first test compound (sometimes referred to as a full product or full product compound), a building block(s) of the first test compound, a first byproduct generated during synthesis of the first test compound (sometimes referred to as a byproduct or side product), or an intermediate generated during synthesis of the first test compound (sometimes referred to as an intermediate), or a combination of two or more thereof. In some embodiments, the first test compound is a desired product of a synthesis reaction comprising two or more of the building blocks of the first test compound. In some embodiments, the first byproduct is an undesired product of the synthesis reaction comprising the two or more building blocks of the first test compound. In some embodiments, one or more compounds of the second library (or optionally subsequent library as applicable in an iterative method) is a second test compound (sometimes referred to as a full product or full product compound), a building block(s) of the second test compound, a second byproduct generated during synthesis of the second test compound (sometimes referred to as a byproduct or side product), or an intermediate generated during synthesis of the second test compound (sometimes referred to as an intermediate), or a combination of two or more thereof. In some embodiments, the second test compound is a desired product of a synthesis reaction comprising two or more of the building blocks of the second test compound. In some embodiments, the second byproduct is an undesired product of the synthesis reaction comprising the two or more building blocks of the second test compound. In some embodiments, one or more compounds of the third library is a third test compound (sometimes referred to as a full product or full product compound), a building block(s) of the third test compound, a third byproduct generated during synthesis of the third test compound (sometimes referred to as a byproduct or side product), or an intermediate generated during synthesis of the third test compound (sometimes referred to as an intermediate), or a combination of two or more thereof. In some embodiments, the third test compound is a desired product of a synthesis reaction comprising two or more of the building blocks of the third test compound. In some embodiments, the third byproduct is an undesired product of the synthesis reaction comprising the two or more building blocks of the third test compound. In some embodiments, the full product comprises a trisynthon and the intermediate product comprises a disynthon and/or monosynthon.

In some embodiments, the first compound descriptor comprises data or information associated with the compound of the first library of compounds, wherein the data or information comprises binding affinity of the compound to a target molecule (optionally wherein the target molecule is a drug target such as a protein or a nucleic acid), activity of the compound, a physical property of the compound (e.g., lipophilicity), toxicity of the compound, stability of the compound, permeability of the compound, sequencing reads associated with an abundance of the compound in an experiment, compound structure, information related to synthesis of the compound, labeling data, process quality control data, yield associated with synthesis of the compound, sequencing data, labeling data, product data, synthesis efficiency data, mass spectrometry data, compound fraction data, binding data, matrix binding data, promiscuity data, structure data, or building block validation data, or a combination of two or more thereof. In some embodiments, the second compound descriptor comprises data or information associated with the compound of the second library of compounds, wherein the data or information comprises binding affinity of the compound to a target molecule (optionally wherein the target molecule is a drug target such as a protein or a nucleic acid), activity of the compound, a physical property of the compound (e.g., lipophilicity), toxicity of the compound, stability of the compound, permeability of the compound, sequencing reads associated with an abundance of the compound in an experiment, compound structure, information related to synthesis of the compound, labeling data, process quality control data, yield associated with synthesis of the compound, sequencing data, labeling data, product data, synthesis efficiency data, mass spectrometry data, compound fraction data, binding data, matrix binding data, promiscuity data, structure data, or building block validation data, or a combination of two or more thereof. In some embodiments, the compound is a full product compound, intermediate product compound, or byproduct compound.

In some embodiments, the method comprises testing one or more of the compounds of the first library of compounds in an in vitro or in vivo assay. In some embodiments, the method comprises testing one or more of the compounds of the third library of compounds in an in vitro or in vivo assay.

In some embodiments, each compound of the third library of compounds comprises or is synthesized to comprise a nucleic acid tag, the method further comprising sequencing the third library of compounds to generate sequencing data associated with the third library of compounds. In some embodiments, the information of step (b) comprises sequencing data associated with an external library of compounds (e.g., a library comprising nucleic acid tags from each compound in the first library and/or second library). In some embodiments, the compound fitness score for each compound in the first library of compounds is generated from data comprising sequencing data associated with the first library of compounds. In some embodiments, the sequencing data comprises a read count, a quality score associated with the read count, and/or comprises a score calculated from the sequencing read count or set of read counts from different experimental conditions from the first library of compounds and/or the second library of compounds. In some embodiments, the score comprises the read count or the read counts divided by the total number of reads in a selection of compounds or the average number of reads in a selection of compounds, or similar a mathematical function that has utilized a read count (directly or indirectly).

In some embodiments, at least one compound fitness score for each compound of the first library of compounds is generated from data comprising a first compound descriptor (e.g., sequencing data, labeling data, product data, synthesis efficiency data, mass spectrometry data, compound fraction data, binding data, matrix binding data, promiscuity data, structure data, or building block validation data, or a combination of two or more thereof). In some embodiments, the prediction model utilizes a probabilistic framework to process the first data set and the second data set, and to output the compound fitness score for each compound of the second library of compounds. In some embodiments, the fitness score is generated at least in part from data from a full product compound comprising a non-target count, a target count, and/or a product proportion adjustment value. In some embodiments, the fitness score is generated at least in part from data from an intermediate product compound comprising a no target control count, a target count, and/or a product proportion adjustment value. In some embodiments, the method comprises generating a compound fitness score for each compound in the third library of compounds utilizing sequencing data associated with sequencing the third library of compounds.

In some embodiments, the method comprises assaying the third library of compounds. In some embodiments, the assay comprises binding the third library of compounds to a target. In some embodiments, the assay comprises sequencing the third library of compounds or a subset of the third library of compounds (e.g., wherein the subset is a subset of compounds that binds to the target).

In some embodiments, the fitness score of any one of the compounds comprises a binding and/or activity score of the compound. In some embodiments, the third library comprises one or more compounds from the second library with a compound fitness score greater than a threshold score.

In some embodiments, the method comprises pre-processing the first data set and/or the second data set. In some embodiments, the pre-processing step is performed before step i and/or before step iii of the computer implemented method. In some embodiments, the method comprises refining a fitness score generated from the prediction model, optionally wherein the refinement is performed by the prediction model, and/or optionally wherein refining comprises incorporating information from an external library (e.g., a library of nucleic acid tags associated with the first library and/or second library of compounds).

In some embodiments, the second library comprises one or more different compounds than the first library. In some embodiments, the second library comprises one or more compounds different from the first library.

In some embodiments, the method comprises repeating steps ii-vi to update the model. In some embodiments, steps (a)-(b) are iteratively repeated to identify a set of potential compounds with one or more desired properties. In some embodiments, steps (a)-(b) are iteratively repeated to identify a set of potential compounds with one or more desired compound fitness scores.

In some embodiments, a compound fitness score is in relation to an oral drug solubility, intestinal absorption, a permeability, a hERG toxicity, a CYP inhibition, a blood brain barrier permeability, a P-glycoprotein activity, plasma protein binding, and/or a binding affinity of any one of the compounds.

In some embodiments, the first compound descriptor input into the model comprises compound structure and/or experimental data. In some embodiments, the prediction model is a machine learning model.

In some embodiments, the machine learning model comprises a neural network. In some embodiments, the neural network is a graph neural network. In some embodiments, the machine learning model comprises a graph neural network and an attention layer. In some embodiments, the neural network is a graph attention network.

In some embodiments, the method comprises performing a validation assay on at least one compound of the third library of compounds. In some embodiments, the method comprises performing low-throughput analysis on at least one compound of the third library of compounds. In some embodiments, the method comprises inputting a third data set comprising a compound descriptor and a compound fitness score for each compound of the third library of compounds into a secondary system in a validation assay. In some embodiments, the method further comprises inputting data from the validation assay into the prediction model. In some embodiments, the validation assay comprises a proxy for binding or biochemical activity including one or more of absorbance, fluorescence, luminescence, radioactivity, NMR, crystallography, microscopy including cryo-electron microscopy, mass spectrometry, or Raman scattering, for example, Surface Plasmon Resonance (SPR) measures the reflection of polarized light, which can detect the change in the reflection angle (refractive index), and immobilization or binding of a ligand (compound) to the surface (which contains the immobilized target protein) affects the mass or thickness of the surface, which changes the refraction.

In some embodiments, the prediction model generates a predictive compound descriptor for each compound in the first library of compound and/or the second library of compounds. In some embodiments, the compound fitness score is generated at least in part from the predictive compound descriptor for each of the compounds.

In some embodiments, the first library of compounds is about 10,000 compounds to about hundred billion compounds or about 10,000 compounds to about ten billion compounds; and wherein the second library of compounds is about 10,000 compounds to about hundred billion compounds or about 10,000 compounds to about ten billion compounds.

In one aspect, provided herein is a computer-implemented system comprising: a digital processing device comprising: at least one processor, an operating system configured to perform executable instructions, a memory, and a computer program including instructions executable by the digital processing device to perform a method described herein.

In one aspect, provided herein is a non-transitory computer-readable storage media encoded with a computer program including instructions executable by one or more processors to perform a method described herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a flow chart of a method of iteratively screening a DNA-encoded library of compounds for target binding affinity and using the data to train a machine learning model to generate predictions of compounds for an updated library of compounds, per one or more embodiments disclosed herein;

FIG. 2 shows an illustration of three synthetic steps in a combinatorial chemical pathway for library generation, per one or more embodiments disclosed herein;

FIG. 3 shows a comparison between a traditional compound screening process and DNA-encoded libraries allowing for large-scale screening;

FIG. 4 and FIG. 5 show an illustration of a pool and split process for further expanding the size of a DNA-encoded library;

FIG. 6 shows an illustrative process for a binding assay experiment to identify compounds that bind to a target molecule within a combinatorial library of DNA-encoded compounds;

FIG. 7 shows an illustrative example of data from a binding assay experiment that can be converted into input for a machine learning model to generate a representation of binding interactions between compounds and a target molecule;

FIG. 8 shows an illustrative graph mapping the compounds in a library to known products, known intermediates, and unknown compounds, which can be used for quality control;

FIG. 9 shows mass spectrometry data of compounds in a reaction mixture before and after the chemical synthetic step takes place, which can be used to determine synthesis efficiency for quality control purposes;

FIG. 10 shows reference mass spectrometry data of the library region;

FIG. 11 shows the compound fractions that correspond to the series of chemical synthetic steps used to construct a DNA-encoded library;

FIG. 12 shows a graph of sequencing read count plotted against the number of compounds as compared between the DNA-encoded library disclosed herein and a traditional DEL in which the DNA-encoded library disclosed achieves higher dynamic range by having the same sequencing depth for a small number of molecules;

FIG. 13 provides an overview of the iterative DEL library process using the deep neural network architecture as disclosed herein;

FIG. 14 provides an illustration of the graph convolutional neural network architecture configured to receive input data and generate a predicted compound fraction and binding score, in accordance with one or more embodiments herein;

FIG. 15 shows a star chart plotting c Log P, Molecular Weight (MW), Hydrogen Bond Acceptors (HBA), Hydrogen Bond Donors (HBD), Rotatable Bonds (RB), Topological Polar Surface Area (TPSA), and SP3 Fraction (fSP3);

FIG. 16 shows a non-limiting example of a computing device; in this case, a device with one or more processors, memory, storage, and a network interface;

FIG. 17 shows a non-limiting example of a web/mobile application provision system; in this case, a system providing browser-based and/or native mobile user interfaces;

FIG. 18 shows a non-limiting example of an application provision system alternatively as a distributed, cloud-based architecture;

FIG. 19 shows a comparison of compound rankings based on predicted binding scores generated by an initial baseline model and an updated model with an evolved DNA-encoded library as validated according to true positive hits;

FIG. 20 shows a multiparameter score comparison between compounds in the initial DNA-encoded library and compounds identified in a first evolved DEL using a first evolved model (ML1) after a first iteration and compounds identified in a second evolved DEL using a second evolved model (ML2) after a second iteration;

FIG. 21 shows an evaluation of predicted compound binding with respect to two highly related protein domains (95% sequence similarity) within a single protein with the compound score for Domain 1 plotted on the Y axis and the compound score for Domain 2 plotted on the X axis. Each compound is represented by a blue dot, and the scores are directly derived from the DEL data; and

FIG. 22 shows an evaluation of predicted compound binding with respect to two related proteins within the same class of chromatin regulators in which a number of compounds were predicted to specifically bind only one of the two proteins; and

FIG. 23 shows a non-limiting example of a model architecture for predicting enrichment and adjustments values of trisynthon and disynthons;

FIG. 24 shows bar graphs illustrating Loss and R² scores on the test data for a full ML model compared to other models;

FIG. 25 shows a bar graph illustrating the area under the ROC curve for 150 external molecules tested using the full ML model and various other models; and

FIG. 26 shows a bar graph illustrating the Hit rate on top 10 of 150 external molecules tested using the full ML model and a flat yield model.

### DETAILED DESCRIPTION

Disclosed herein are platforms, systems, and methods for carrying out compound discovery with DNA encoded chemical libraries (referred to as “DEL”) in an iterative process using artificial intelligence. Compound discovery can involve an integrated process that combines DEL design, experimentation, computational/ML analysis, and follow-up experimentation to enable rapid feedback loops between the experimentation and the computation. The integration of the entire iterative process end-to-end allows generation of higher quality data at a greater scale, flexibility, specificity, and speed. In addition, the ML-assisted iteration of DELs can include dynamic reconfiguration of building block selection with each iteration, which generates richer and more useful data. The ML model may then choose building blocks and synthetic schemes that have little to no overlap with the original library. This may help ensure that only the best compounds are built, even if the compounds could not have been tested in the source libraries. In some cases, library production is more flexible and may allow for more varied library synthesis schemes. For example, library designs, synthetic schemes and production methods that would otherwise be uneconomical, but may generate high value structures, become viable because ML-guided synthesis decisions are more likely to succeed and allow more focused effort.

### DNA Encoded Library

A DEL is a collection of chemical compounds, which can be (but not required to be) stored in a single tube, in which the compounds in the tube are each physically linked to a unique DNA sequence that represents the compound (which can be used as a barcode to identify the compound). In some cases, as used herein, the DNA sequence, structure, synthesis or other data or information that is related to the compound, may be referred to as a compound descriptor. In some embodiments a compound descriptor comprises data or information associated with the compound of a library of compounds. The data or information may comprises binding affinity of the compound to a target molecule (optionally wherein the target molecule is a drug target such as a protein or a nucleic acid), activity of the compound, a physical property of the compound (e.g., lipophilicity), toxicity of the compound, stability of the compound, permeability of the compound, sequencing reads associated with an abundance of the compound in an experiment, information related to synthesis of the compound, labeling data, process quality control data, or yield associated with synthesis of the compound, sequencing data, labeling data, product data, synthesis efficiency data, mass spectrometry data, compound fraction data, binding data, matrix binding data, promiscuity data, structure data, or building block validation data or a combination of two or more thereof. By creating the link between up to billions of compounds and corresponding unique DNA sequences, a single DEL tube effectively miniaturizes compound collections that would otherwise take up entire warehouses.

A DEL may be constructed through combinatorial chemistry in which chemical building blocks (e.g., monomers) are assembled together onto the end of a piece of DNA, thereby synthesizing various encoded combinations in a single tube. Every time a new building block is added, a new segment of DNA is also added, and the DNA acts as a barcode for said building block. Accordingly, the DNA barcode corresponding to the compound grows as new building blocks are added, whereby the sequence of the DNA barcode enables the identity and the order of addition of the building blocks to be decoded. When given a DNA sequence from a DNA encoded library, one can determine what compound was made (i.e., compound structure) using a key mapping DNA barcodes to building blocks.

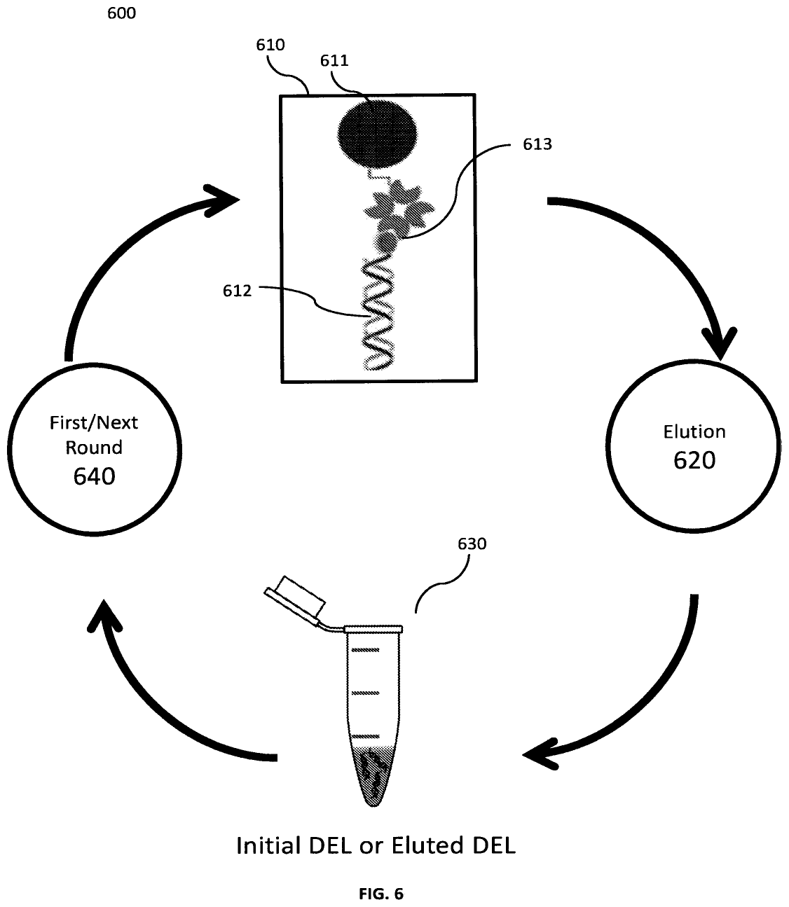

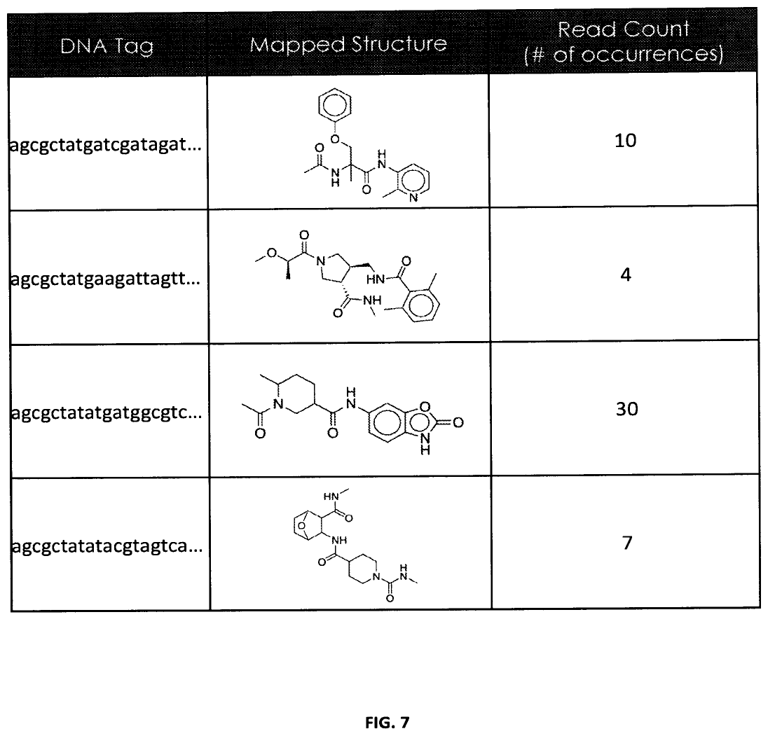

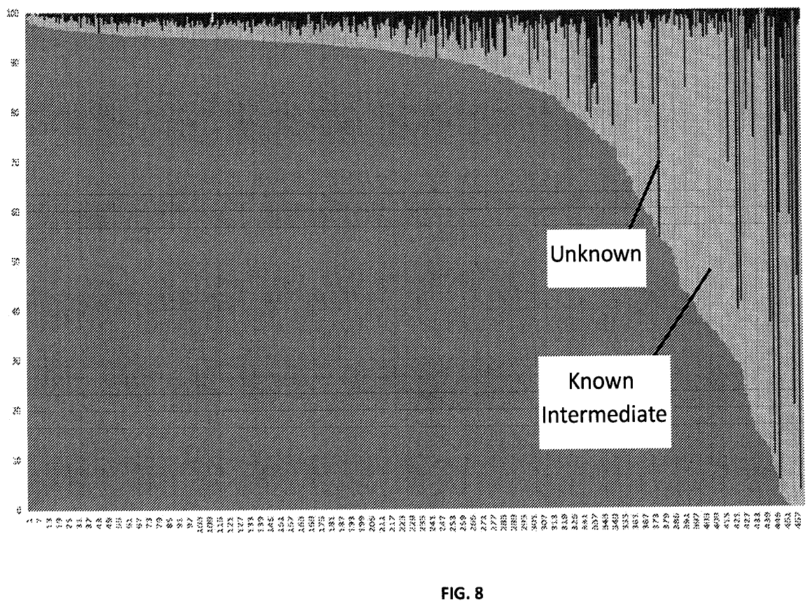

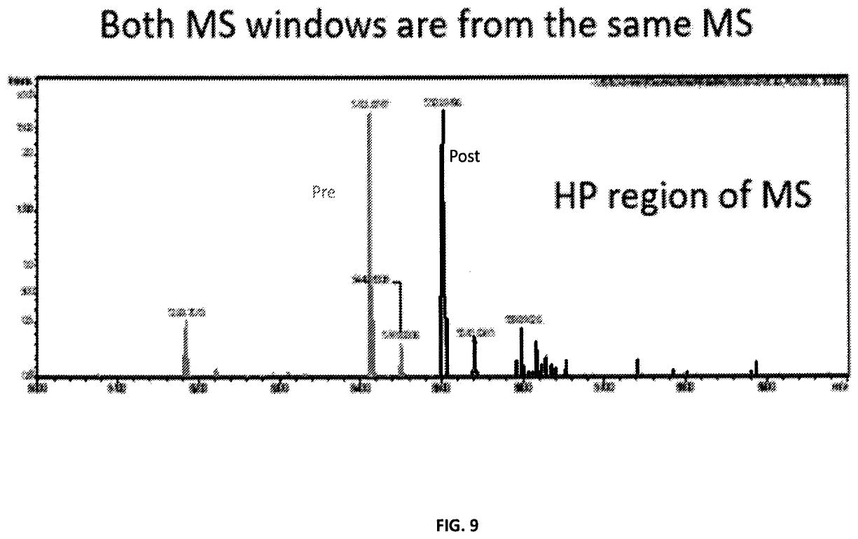

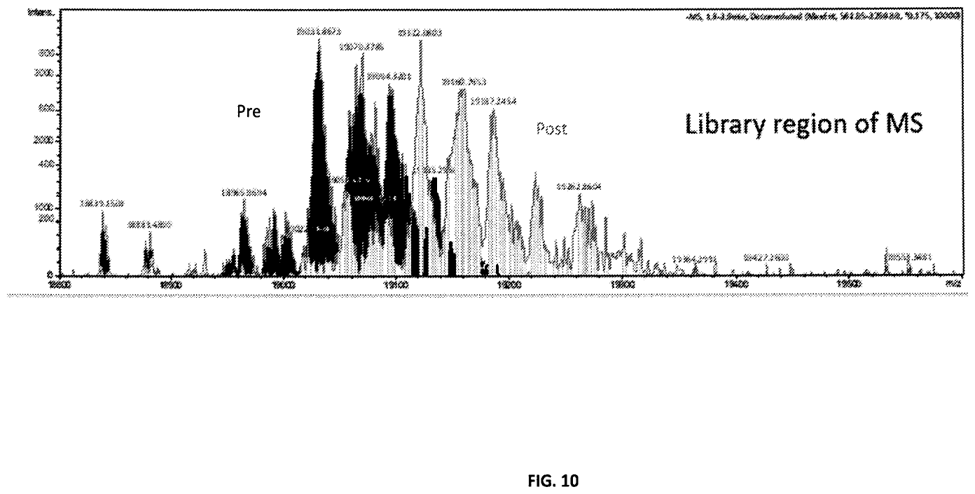

As illustrated in FIG. 6, A traditional HTS segregates individual compounds as usually one compound per well, spatially “labeling” the compounds with their well location. Each of those compounds may be tested one at a time, often in an automated manner to check if that compound binds or otherwise interacts with a target. Compounds that do may be chosen for further optimization. This approach is practically limited to low single digits of compounds, and a small number of measurements per compound. In contract a DEL may label each compound with a DNA sequence that may be decoded later on with a DNA sequencer. This allows for compounds to be mixed in a single tube without compromising the ability to identify each compound later. A compound mixture may be filtered by exposing the mixture to an immobilized protein as illustrated in FIG. 6 and seeing which DNA sequences are recovered as binders to a target. This may allow for billions of compounds to be tested in parallel for hundreds of different scenarios (i.e., different proteins or different versions of proteins). This help generate large volumes of dense data for machine learning purposes.

In some embodiments, the DEL library is configured with a size within an optimized range. One of the advantages of a DEL library over high-throughput screening is that it can screen for hundreds of millions of compounds instead of a few million. In some cases, the DEL library has at least 100 million, 200 million, 300 million, 400 million, 500 million, 600 million, 700 million, 800 million, 900 million, 1 billion, 2 billion, 3 billion, 4 billion, 10s of billions of unique encoded compounds.

However, while a larger DEL library can theoretically allow testing of a larger chemical space, an overly large library can include too many non-drug like compounds, poor chemistry fidelity, and inherently poor signal to noise ratio that contaminate downstream analysis with useless and/or uninterpretable data. Several parameters that can govern the DEL signal to noise ratio include the number of copies of each individual molecule at the start of the experiment, the number of rounds of selection that are performed, and the number of sequencing reads a sequencer can produce. In excessively large libraries, each individual compound may be only represented by a few copies within the tube, and many compounds may no longer be present after a few rounds of selection while those that remain may be represented by even fewer copies. The sequencer may only output a fixed number of reads. By starting with more possible compounds, each individual compound only receives a small number of reads, thereby making it difficult to distinguish one compound's performance from another. As a result, DEL libraries can become increasingly noisy as they grow larger, particularly beyond a certain threshold. In some cases, the DEL library has no more than 100 million, 200 million, 300 million, 400 million, 500 million, 600 million, 700 million, 800 million, 900 million, 1 billion, 2 billion, 3 billion, 4 billion, 5 billion, 10 billion, up to or greater than 100 billion unique encoded compounds. In some aspects, the methods and systems herein utilize or comprise multiple DELs, e.g., as an input or output of a model, and as such, each DEL may vary in size.

Moreover, conventional DEL approaches may use relatively smaller libraries to provide a clearer signal, but in doing so they compromise their ability to widely explore chemical space. By contrast, the platforms, systems, and methods disclosed herein can enable the computational design of highly efficient libraries to cover a massive chemical space despite making fewer overall compounds downstream. For example, an initial set of DEL libraries may be constructed and tested for binding to a given target. The resulting dataset may consist of a smaller set of compounds with varying binding affinities to the target and a much larger set of compounds which did not bind appreciably. Due to their small numbers, the compounds that bound to the target in the initial library will necessarily be far less diverse than the initial library. Furthermore, any compounds that are chemically similar (via molecular fingerprints and tanimoto distance for example) to these initial compounds will struggle to expand this relatively narrow chemical space. While smaller libraries may make it easier to reliably identify useful compounds, those compounds are more limited in number.

In some embodiments, the method of training and the model described herein may solve the inherent limitations explained above by exploiting the higher signal to noise ratio of the dataset to train machine learning (ML) algorithm(s) to build ML models that have a preliminary understanding of the chemical space suitable for binding to a target. The preliminary understanding of the model and the ability to train the model on a dataset that provides a more wholistic description of the compounds of the library (e.g., a DEL) allow the ML models to explore beyond chemical diversity of the original hits and design a new library that may share little overlap in the synthetic plan and/or building blocks (e.g., compounds) present in the original library, wherein the ML model may use binding to a target as a primary criteria. The resulting library may be more structurally diverse compared to an initial positive exemplars and/or their derivatives while being more focused on areas of chemical space rich in compounds binding to the desired target. This enables the ML model to determine which building block(s) and/or library designs are most effective for a given target allowing the ML model to more effectively a identify more useful compounds, for example by generating a fitness score for the compounds, while testing fewer total compounds. In some embodiments, one or more compounds (e.g., building blocks) and/or libraries in the ML designed synthesis may be in the initial library. Theoretically a library containing all possible compounds (e.g., building blocks) could be constructed, but that library would be impractically expensive to construct, and would require 100 or 1000 times or more materials as well as 100 or 1000 or more times the sequencing and analysis capacity to achieve the same signal to noise and identify useful compounds.

The platforms, systems, and methods disclosed herein are able to exploit the superior signal of a smaller library combined with a machine learning driven iterative design of smaller libraries to search a chemical space much more efficiently, and may provide a clearer understanding of a chemical space for a given target. Even with the largest practical initial library using conventional methods and models, the ML model trained and described herein is able to help create and use smaller high signal to noise libraries to search for compounds more effectively while still providing a clearer understanding of the chemical space in view of a target. In some embodiments, the signal to noise may be a percentage of compounds that are confirmed to work in a secondary assay among the total compounds tested in that secondary assay.

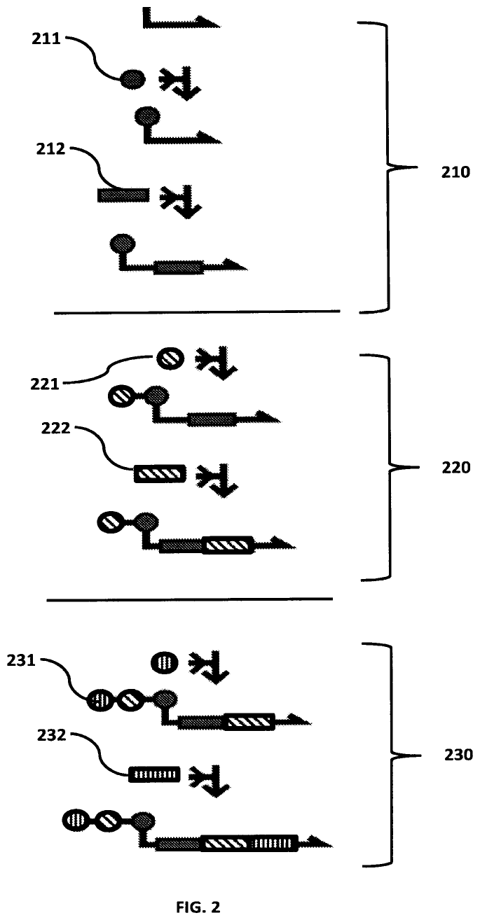

In some embodiments, a library (e.g., a DEL) may be constructed as illustrated in FIG. 2 by adding a building block or compound 211 to a tube containing a substrate. The building block 211 may be coupled to a piece of DNA 212 with a unique sequence representing the building block 211. This is performed during a single synthetic cycle 210. In some embodiments, a second cycle 220 may performed wherein a new building block 221 and DNA tag 222 may be added to the chain from the first cycle 210. In some embodiments a third cycle 230 may be performed a new building block to form a longer chain 231 incorporating a new DNA tag 232 each time. In some embodiments, more than three cycles may be performed and the processes may be repeated until one or more desired DELs are constructed.

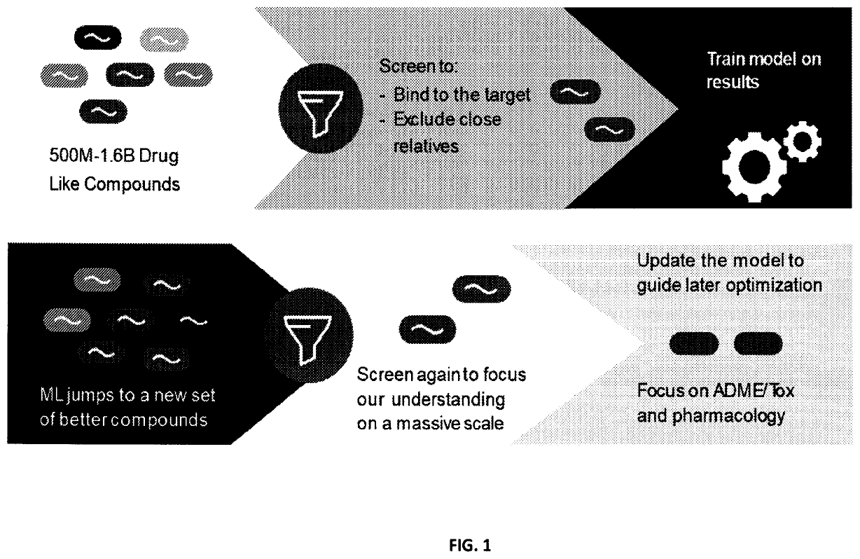

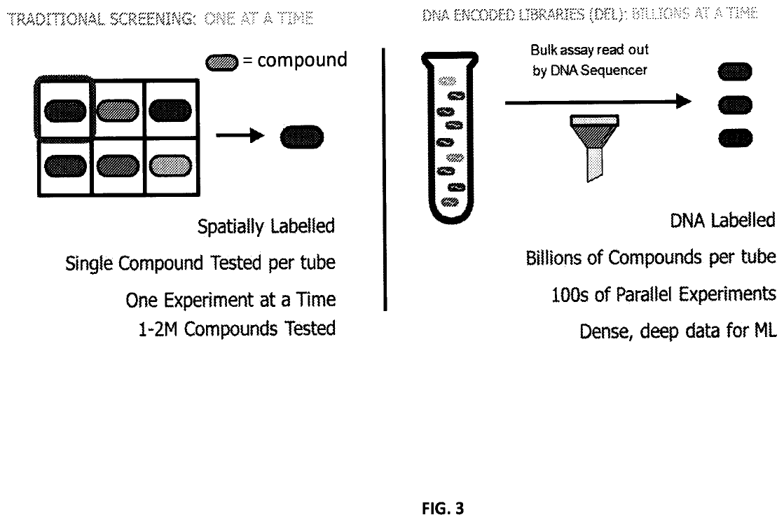

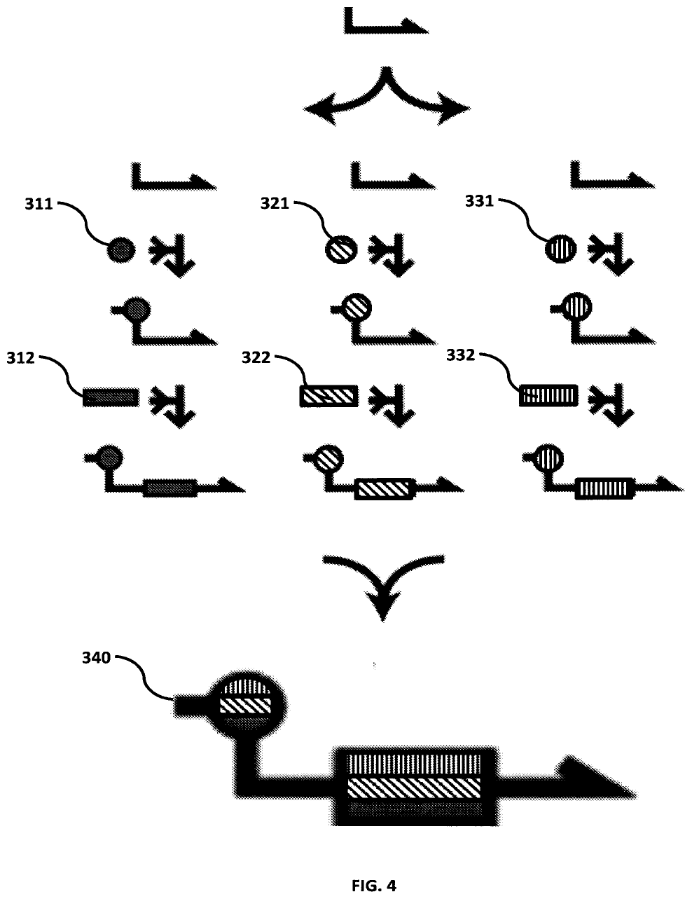

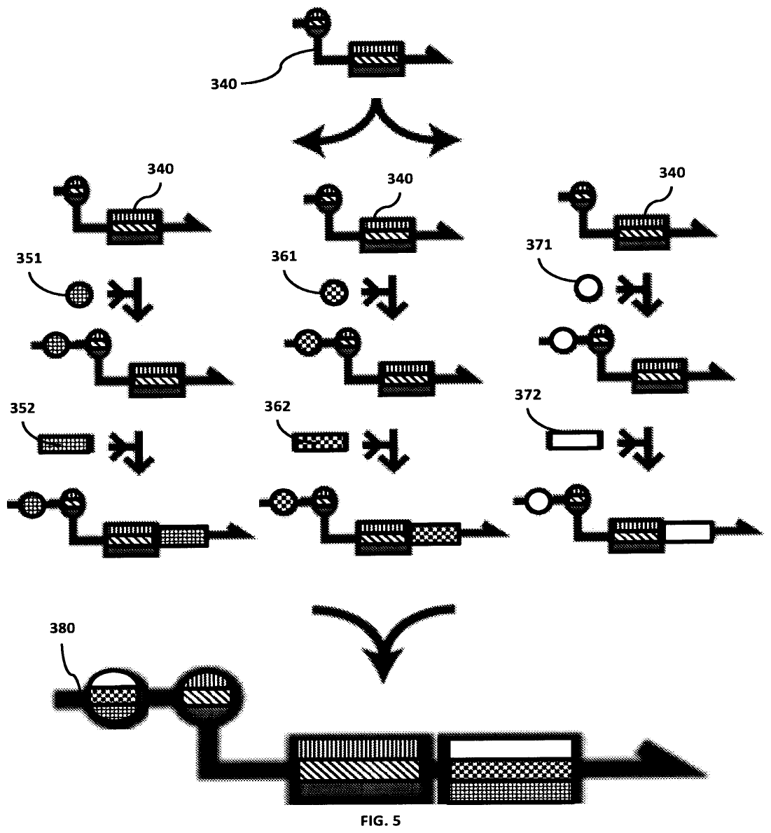

The size of a library (e.g., DEL) may be further expanded using a split pool synthesis method, as illustrated in FIGS. 4-5. In some embodiments, a substrate is split into several tubes, as illustrated in FIG. 3, however more tubes (e.g., splits) may be used. A different building block 311, 321, 331 is added to each tube. A corresponding DNA tag labeling each building block 312, 322, 332 may also be added to each tube. In some embodiments, each tube may go through a single cycle as described in the first step 210 of FIG. 1. The results of each tube may be pooled together to create a mixture 340 of all 3 resulting chemical species.

In some embodiments the split pool DEL synthesis method may further comprise a second step, as illustrated in FIG. 5. The mixture of compounds 340 resulting from the first step illustrated in FIG. 4 may be split into multiple tubes (e.g., 3). In some embodiments more than three tubes may be used. A different building block 351, 361, 371 may be added to each tube. A corresponding DNA tag labeling each building block 352, 362, 372 may also be added to each tube. In some embodiments, each tube goes through a second cycle as described in the second step 220 of FIG. 1. The results may be pooled together to create a mixture 380 of all 3 resulting chemical species. As illustrated in FIGS. 4-5 by having a starting point be mixture 340, the resulting mixture 380 of the second step may be a combination of all possible building blocks at both a first and second position. This results in 3*3=9 different compounds in the mixture 380. In some embodiments the process can be repeated additional times to create more complex mixtures. For example, an addition step would result in 3*3*3=27 compounds.

Library construction (e.g., DEL construction) may include one or more of several construction techniques such as: building-block combinations, scaffold-directed (i.e., modifying core scaffolds), or in-situ ring formation (e.g., forming diverse heterocycles). In some embodiments, a DEL library is constructed to be greater than a threshold percentage compliant with Lipinski's Rule of 5 (a rule of thumb for evaluating a compound as a candidate drug). Specifically, Lipinski's Rule of 5 requires: a maximum of 5 hydrogen bond donors (NH and OH bonds); a maximum of 10 hydrogen bond acceptors (nitrogen or oxygen atoms only); and molecular mass less than 500 Da; octanol-water partition coefficient (log P) that is no more than 5. In some cases, the DEL library is constructed to be greater than 60%, 65%, 70%, 75%, or 80% Rule of Five compliant. In some embodiments, the library is screened to ensure that no more than a threshold percentage of compounds are unknown. The compound (e.g., building block) used to generate a DEL or portion thereof may be evaluated to determine the amount of desired product, the amount of known intermediates and byproducts, and the amount of unknown. In some embodiments, a building block will not be passed into production for DEL construction when the percentage of unknown compounds exceeds a threshold amount. For example, the building block may fail the test if the percentage of unknown compounds exceeds 1%, 2%, 3%, 4%, 5%, 6%, 7%, 8%, 9%, 10%, 11%, 12%, 13%, 14%, 15%, 16%, 17%, 18%, 19%, or 20% or more.

In some embodiments, extensive in-library quality control may be conducted. In a non-limiting example, a spike-in may be conducted to monitor an un-ligatable headpiece substrate into every well of synthesis to monitor a single conversion on the lower molecular weight headpiece region of the molecule, which may be used to give better confidence on the higher molecular weight regions when small molecular weight transformations can be difficult to monitor on large 30+ kDa molecules.

A DEL allows for bulk assays to be performed as opposed to individual screening, which is more time and resource intensive. In a non-limiting example of process 600 for isolating compounds of interest, a DEL 630 containing billions of compounds may be provided within a single tube or well for a target binding assay. For example, a protein of interest 613 (e.g., a “target”) may be immobilized on a support 611, and the entire DEL 630 is incubated with that immobilized protein 613, which allows compounds 612 from the DEL 630 to bind to the protein 613 and/or the support 611 to form a resulting complex 610. The complex 610 may be washed (i.e., with a buffer) to remove supports and/or unwanted weak binders or non-specific binders. The remaining compounds may be eluted 620 to release the binding compounds 612 that bound to the protein 613. The process 600 may be repeated with the eluted compounds and reapplied for another round 640. A DNA tag within a resulting mixture may be amplified (e.g., via PCR) and sequenced, giving a list of sequences (representing compound structures) that bound to the target protein, and how often those sequences were represented in the tube. Since the compounds (e.g., building blocks) and their corresponding DNA segments are predetermined, the compounds bound to the protein may be identified by the sequences detected in the bound compound. The compounds that successfully bound to the target protein (and not to a negative control such as the support) may be suitable candidates as starting points for drug design.

In some embodiments, a DEL experiment may seek to identify which compounds in a library are binding to a protein of interest. A DEL experiment may entail one or more experimental conditions (e.g., each tube can be seen as a mini-experiment or “condition”). In some embodiments, the results of a condition tested in DEL experiment may be a compound descriptor. In some embodiments, a simple DEL experiment may include two conditions: a binding assay with the protein of interest and the other binding assay with no protein that serves as a control. Because a condition tests the entire DEL of compounds, each condition creates a massive dataset of its own for millions or even billions of compounds. In some embodiments, the dataset may be a compound descriptor. In some embodiments, the dataset may be a used to generate a compound fitness score. This means that DELs miniaturize a massive experiment into a single tube at low cost, in contrast to other approaches such as high throughput screening (HTS) that conduct individualized assays on a massively parallel scale. In a non-limiting example, instead of investigating a single condition, DELs allow an experiment to be conducted across multiple conditions to ask a number of questions about a compound, for example information about or properties of the compound (sometimes referred to as a compound descriptor), including but not limited to: affinity (at high or low concentrations of protein), specificity (against a mutated version of the protein or a closely related protein that might be a member of the same protein family as the target), and/or binding location (mutating known binding pockets or adding known competitive binders to the mixture to a target). In some embodiments a compound descriptor comprises data or information associated with the compound of a library (e.g., a DEL) of compounds. The data or information may comprises binding affinity of the compound to a target molecule (optionally wherein the target molecule is a drug target such as a protein or a nucleic acid), activity of the compound, a physical property of the compound (e.g., lipophilicity), toxicity of the compound, stability of the compound, permeability of the compound, sequencing reads associated with an abundance of the compound in an experiment, information related to synthesis of the compound, labeling data, process quality control data, or yield associated with synthesis of the compound, sequencing data, labeling data, product data, synthesis efficiency data, mass spectrometry data, compound fraction data, binding data, matrix binding data, promiscuity data, structure data, or building block validation data or a combination of two or more thereof.

In some embodiments, each condition may be run in its own tube before being read out on a sequencer. The number of conditions used in an experiment may be at least 2, 3, 4, 5, 6, 7, 8, 9, or 10 or more conditions. The conditions may include different protein concentrations such as at least 2, 3, 4, 5, 6,7, 8, 9, or 10 or more protein concentrations. The conditions may include different mutations of the target protein such as at least 2, 3, 4, 5, 6,7, 8, 9, or 10 or more different mutations. The conditions may include one or more internal controls such as at least 1, 2, 3, 4, 5, 6, 7, 8, 9, or 10 or more internal controls. In some embodiments, the conditions may include an internal control for each experimental condition. In a non-limiting example being, a binding assay for a wild-type protein may include internal controls including (1) the support (no protein), (2) a different protein not expected to produce specific binding (e.g., bovine serum albumin), and (3) the protein with a mutation to the binding pocket.

DEL data may be a series of sequences of DNA and the number of times that sequence was observed. The sequence may be decoded into a compound structure. The number of times a structure is observed (e.g., the number of observations of the sequence or “hits”) may be related to how tightly that structure bound to the target. In some embodiments, a compound fitness score is in relation to the number of “hits”. However, the number of hits may also be related to various factors before and after the binding event. These factors may include library production efficiency, PCR amplification, sequencing and many other steps that can affect the count and/or a compound fitness score. For example, the synthesis steps during library production may have different efficiencies that resulted in an unequal number of compound species within the library. A compound that was synthesized at a lower efficiency may then yield a relatively lower number of sequence “hits” than would be expected based on binding efficiency simply because there was a smaller amount of the compound within the library.

The conventional DEL dogma holds that the output of a DEL cannot be correlated to the affinity that compound has for the target. A compound might show up as very enriched (i.e., has many reads in the sequencer) in a DEL selection, but only be a weak binder and vice versa. This is because the DEL readout is a noisy process. Many factors complicate the readout which can including, for example, variable chemistry yield, unexpected side products generated during library synthesis, DNA binding (by the barcode), matrix/support binding, promiscuous binding, under-sampling of compounds, amplification bias, and sequencing noise.

The platforms, systems, and methods disclosed herein may account for sources of noise to yield DEL output with read counts that correlate with binding affinity. In some embodiments, the DELs are built from the ground up to minimize noise and maximize signal, which allows for machine learning models to be developed using the data generated from the DELs to effectively distinguish tighter binders from weaker ones.

### Sequencing & Selection

In some aspects, the platforms, systems, and methods disclosed herein utilize deep sequencing for complete sequencing and not partial reads. In some embodiments, the selection coverage of the molecules in the DEL is at least 50%, 55%, 60%, 65%, 70%, 75%, 80%, 85%, 90%, 95%, 99%, or 100%. In some embodiments, the selection coverage ranges from 50%, 55%, 60%, 65%, 70%, or 75% to 80%, 85%, 90%, 95%, 99%, or 100%. Machine learning may be used to screen libraries of compounds to predict a new evolved library of compounds for an iterated round of analysis. Multiple rounds of selection can be carried out to identify binders.

### Target Molecule

Disclosed herein are platforms, systems, and methods for predicting compounds that interact with a target molecule. The target molecule may be a protein or nucleic acid structure such as a mammalian target protein (e.g., in humans) or a non-mammalian target (e.g., in the case of target molecules that are present in bacteria, yeast, fungi, or parasites) or such as the mRNA transcript of a gene (for example MYC). The target molecule may be associated with a disease or symptom thereof. In some embodiments, the target molecule may belong to a biological pathway associated with a disease or symptom thereof, or modulates or regulates that pathway. Accordingly, a compound that is predicted to bind the target molecule may act to inhibit the target, thereby modulating the associated pathway. In a non-limiting example, a compound may bind to a binding pocket of the extracellular ligand-binding domain of a receptor tyrosine kinase, thereby serving as an inhibitor of receptor-ligand binding.

In some embodiments the target may be a target proteins The protein may be a wild type (i.e., the most common allele in the population) or mutant allele. The mutation may be naturally occurring or an engineered mutation such as mutating a target protein's binding pocket to assess specificity of binding compared to a wild type control condition. Mutations may be silent mutations with no effect on protein function, or they may result in gain-of-function (e.g., enhanced ligand binding) or loss-of-function (e.g., reduced or complete loss of ligand binding).

In some embodiments, the target protein may be an enzyme that catalyzes a chemical reaction. The enzyme may have a globular structure with an active site configured for substrate binding and catalysis that is composed of a relatively small number of amino acid residues. The enzyme may also have an allosteric binding site to which an effector molecule binds to alter the structural conformation of the enzyme, thereby enhancing or decreasing its enzymatic activity. In some embodiments, the target protein is a structural protein. The structural protein may be a fibrous proteins (e.g., collagen) or globular proteins (e.g., actin or myosin). The target protein may be involved in cell signaling such as a transmembrane receptor protein kinase. The target protein may be a transport protein such as a transmembrane ion channel protein. In some cases, the target protein may have a quaternary structure composed of two or more protein subunits. Accordingly, experimental conditions can be conducted to identify compounds that specifically bind to structural features of a particular protein by comparing binding with targeted mutations such as in the substrate binding site or the protein-protein interface between subunits within a quaternary structure.

### Screening and Validation

The compounds identified by the platforms, systems, and methods disclosed herein may be validated through additional screening. Various established techniques may be used to validate individual compounds for target binding. For example, known binding assays can utilize one or more of absorbance, fluorescence, luminescence, radioactivity, NMR, crystallography, microscopy including cryo-electron microscopy, mass spectrometry, or Raman scattering. For example, Surface Plasmon Resonance (SPR) measures the reflection of polarized light, which can detect the change in the reflection angle (refractive index). The immobilization or binding of a ligand (compound) to the surface (which contains the immobilized target protein) affects the mass or thickness of the surface, which changes the refraction. Other methods, for example Fluorescence Resonance Energy Transfer (FRET) measure a change in fluorescence intensity as the target and its ligand come together, with a change in intensity being correlated with that interaction being disrupted by a compound.

### Machine Learning Algorithms

Disclosed herein at platforms, systems, and methods that provide an iterative process where lab experiments and machine learning reinforce one another to generate new data and learn what compounds are likely to interact with a medically relevant target. In some embodiments, machine learning algorithms are utilized to determine a compound property such as binding. In some embodiments, the machine learning algorithms herein employ one or more forms of labels including but not limited to human annotated labels and semi-supervised labels. In some embodiments, the machine learning algorithm utilizes regression modeling, wherein relationships between predictor variables and dependent variables are determined and weighted.

Examples of machine learning algorithms may include a support vector machine (SVM), a naïve Bayes classification, a random forest, a neural network, deep learning, or other supervised learning algorithm or unsupervised learning algorithm for classification and regression. The machine learning algorithms may be trained using one or more training datasets.

In some embodiments, the platforms, systems, and methods disclosed herein provide an efficient search of chemical space with large, experimentally tested datasets in which machine learning is used to interpret the results. In some embodiments, specialized machine learning models are built to leverage that data. In some cases, the model architecture exploits one or more unique features of DEL library construction and represents those features explicitly. This allows the models to outperform more conventional architectures when applied to the same data. Conventional models typically receive as input only the data associating sequence to counts. By contrast, disclosed herein are models that receive data associating sequence to counts and also additional features such as the matrix binding data, the promiscuity data, and/or building block validation data (which estimates what fraction of the reaction will proceed to the next step). In particular, the incorporation of an estimate of the reaction efficiency via the fraction of reaction predicted to proceed to the next step addresses a technical problem in which variations in reaction efficiency can result in poor predictive accuracy when the models assume full reaction yield.

Conventional approaches only use large datasets in their initial search, and rapidly collapse their search space to a few dozen compounds a short time such as weeks or months afterwards. This means the search within chemical space is slow and inefficient because many candidate compounds with potential clinical relevance may be screened out, with the remaining compounds being too few in number to efficiently identify new unrelated compounds. By contrast, the platforms, systems, and methods disclosed herein provide cutting edge parallel chemistry and biochemistry capabilities that allow the generation and testing of large scale datasets faster than anyone else to refine the understanding of chemical space and find better medicines. In some embodiments, the machine learning-designed DEL is an evolved DEL generated as a follow-up to an initial DEL library. The ML-designed DEL may have a smaller size than conventional DEL approaches while maintaining a much larger size than conventional HTS approaches. For example, an initial library may have a size of a billion compounds, while the evolved ML-designed library may be generated with over one million compounds for a target/problem-specific library.

In addition to machine learning-aided library design, selection strategies may be designed to maximize signal in ML model building. The machine learning's improved ability to understand complex relationships allows more selection conditions to be interpreted in parallel. In a non-limiting, different mutants of a protein may be used as well as closely related family members. The machine learning's improved ability to understand complex relationships allows more selection conditions to be interpreted in parallel. In a non-limiting, different mutants of a protein may be used as well as closely related family members. The software applications, systems, and methods described herein use for training and generating the ML model are specifically designed to process and utilize this additional data to more efficiently interpret the selection conditions. Together, these capabilities allow rapid exploration of chemical space and more efficiently and effectively find answers to difficult drug discovery problems.

After an initial DEL search finds a good starting point, ML-designed DELs may be used to refine the understanding of what makes a “good” compound for a given target, significantly shortening the lead optimization process timeline. The ML designed DELs may be conceived while considering one or more factors (where each factor may be considered a descriptor associated with a compound of the DEL) including but not limited to binding affinity, chemical diversity relative to the initial training set, physico-chemical properties such as solubility and log-D, and predicted ADME (adsorption, distribution, metabolism, excretion), and predicted toxicity properties. Because the ML may consider more compounds and large libraries may be efficiently constructed and tested by the system, the chemical space does not narrow as much compared to traditional approaches considering only dozens to hundreds of compounds per iteration.

Traditional HTS and DEL approaches suffer from a collapsing search space. After running an initial medium scale (500K-2M compound HTS) or large-scale (1B+ compound DEL) search, a few dozen compounds are chosen for follow-up and further experimentation. In other words, traditional HTS and DEL collapse their search space by six to nine orders of magnitude immediately, falling back on the slow, expensive process of lead optimization that is responsible for over one third of drug development costs. This transition from a wide angle view of chemical space to a restricted window of a few dozen compounds represents an enormous inefficiency in the drug discovery process.

In contrast, the platforms, systems, and methods disclosed herein may generate million-compound follow-up libraries (e.g., DELs), keeping that wide-angle view of chemical space while still focusing on the best compounds. The dataset generated reinforce the ML model with yet more data, giving it a refined understanding of what compounds perform well for a given target. The datasets may be a compound descriptor. In some embodiments, multiple iterations may be carried out to continually refine and improve the follow-on ML-generated libraries (e.g., ML generated DELs). The ML generated DELs not only provide the ML model with more useful data, it enables the model to efficiently learn from its own predictions. Each library iteration may consist of compounds for which the ML model has a strong hypothesis. In a non-limiting example, the ML model may consider the compounds in a ML generated DEL to be strong binders, or weak binders or is uncertain about the affinity, but for each compound model makes an explicit prediction (e.g., a compound fitness score) which may then be tested in a gold standard experiment. In some embodiments, a compound fitness score is in relation to an oral drug solubility, intestinal absorption, a permeability, a hERG toxicity, a CYP inhibition, a blood brain barrier permeability, a P-glycoprotein activity, plasma protein binding, and/or a binding affinity of a compounds.

A new set of random compounds, or a set of compounds naively chosen as similar to the original library would not provide as much useful information compared to the ML model seeing and interpreting the result of its own predictions. This is because the random compound lack the initial hypothesis provided by the ML model. Therefore, the combined lab and ML iterative systems and methods described herein allow the method of training and updating the ML model to be much more efficient and effective. In some embodiments, DEL data (e.g., a compound fitness score) and a ML predictive accuracy may be sufficient to warrant the synthesis of a smaller number of compounds for lower-throughput testing. At this stage, the iterative process may have generated a highly performant ML model and diverse, high quality compound structure starting points generated from the initial process. Accordingly, the resulting list of compounds may have fewer liabilities and may have an optimized ML model to assist in searching for a next improvement. In some embodiments, a compound fitness score is related to an oral drug solubility, intestinal absorption, a permeability, a hERG toxicity, a CYP inhibition, a blood brain barrier permeability, a P-glycoprotein activity, plasma protein binding, and/or a binding affinity of a compounds.

In some embodiments, an initial DEL has a starting size that is below a threshold. The initial DEL is a non-ML-iterated DEL. The starting size may be smaller than a conventional DEL and not include all possible compounds that could potentially be evaluated, but instead, is designed to optimize overall diversity of compound structure. Then, once the first DEL has been evaluated (e.g., via a binding assay and sequencing of hits), the experimental data (e.g., descriptor and/or a compound fitness score) may be input into a ML model, which is then used to generate and/or identify the evolved/iterated DEL. The evolved DEL may have a size of at least 100,000, at least 200,000, at least 300,000, at least 400,000, at least 500,000, at least 600,000, at least 700,000, at least 800,000, at least 900,000, at least 1 million, at least 2 million, at least 3 million, at least 4 million, at least 5 million, at least 6 million, at least 7 million, at least 8 million, at least 9 million, or at least 10 million compounds. With each iteration, the DEL may remain the same or decrease in size until a sufficient number of compounds having a threshold quality (e.g., binding score, affinity score, activity score, compound fitness score, etc.) have been identified for follow-up low-throughput analysis.

The use of an initial library (e.g., DEL) that is below a threshold size (e.g., below 100 billion, 10 billion, 1 billion, 500 million, 100 million, etc.) may significantly improve the signal to noise ratio. Larger libraries suffer from too high noise for too little signal, which means that the same compounds in a larger DEL would be harder to find due to the noise, much less comparing those compounds to others in the library. In some embodiments, evolved libraries (e.g., an evolved DELs) disclosed herein may use specialized building blocks with more interesting structures that provide increased structural diversity, and therefore help increase the chemical space that is searched to identify hits that have potential (e.g., specific target binding). The follow-up evolved DEL may then include new compounds not previously included in the initial DEL, but which may be structurally predicted by the ML model to result in binding and/or enhanced binding. In this way, the wide breadth of the chemical space search is balanced with selectivity for the desired property and ability to reliably identify compounds among the noise.

Various machine learning models and algorithms may be utilized according to the platforms, systems, and methods disclosed herein. Machine learning methods may include Artificial Neural Network, Decision Tree, Support Vector Machine, Regression Analysis, Naïve Bayes, Random Forest, Gradient Boosting, XGBoost, and other suitable techniques. For example, the model architecture may include XGBoost on molecular features, multilayer perceptron on fingerprint vectors, or graph convolutional neural network (GCNN). In some embodiments, the machine learning model comprises a neural network. The neural network may be a convolutional neural network (CNN). In some embodiments, the machine learning network comprises a graph convolutional neural network (GCNN). GCNNs are well suited to evaluating the graph type data representation of molecules. The sequence may be converted to a set of structures that are represented with a graph with arrays associated to each node and edge in the graph.

In some embodiments, a neural network architecture comprises one or more sequence-oriented layers to account for a sequence element of DELs (each compound is the product of a series of reaction steps). The ability to add these sequence-oriented layer(s) is a unique feature of neural networks that have been leveraged to provide additional useful information to the model for improved compound selection to generate the evolved/iterated DEL. In a non-limiting example, these sequence layers may be used to show the ML model a set of compounds that might have been synthesized during the inherently noisy process of library construction. In contrast, a traditional architecture only consider a single compound at a time, and therefore are unable to consider this complexity. The sequence layers may also incorporate additional information, including but not limited to synthetic yield of various reactions, enabling the ML model to better learn a relative importance of different compounds within the set it is shown. The architecture of the ML model described herein, enables incorporation of one or more new and different types of data into the training dataset, enabling the ML model trained on the data to provide more accurate and better compound design.

In some embodiments, a DEL is assayed according to a selection experiment having one or more conditions. For example, the DEL may be evaluated in a protein binding assay against a target protein under one or more conditions (and one or controls), and the bound compounds are then sequenced to determine their corresponding DNA code for identification. The sequencing data may comprise the unique DNA sequences detected, and their corresponding number of sequence counts or hits, which may correspond to a relative abundance of the compounds within the sequenced sample (notwithstanding the various sources of noise discussed herein). The detected compounds may include the entire structure or a portion of the structure such as monosynthon, disynthon, trisynthon, side-products or other polysynthon.

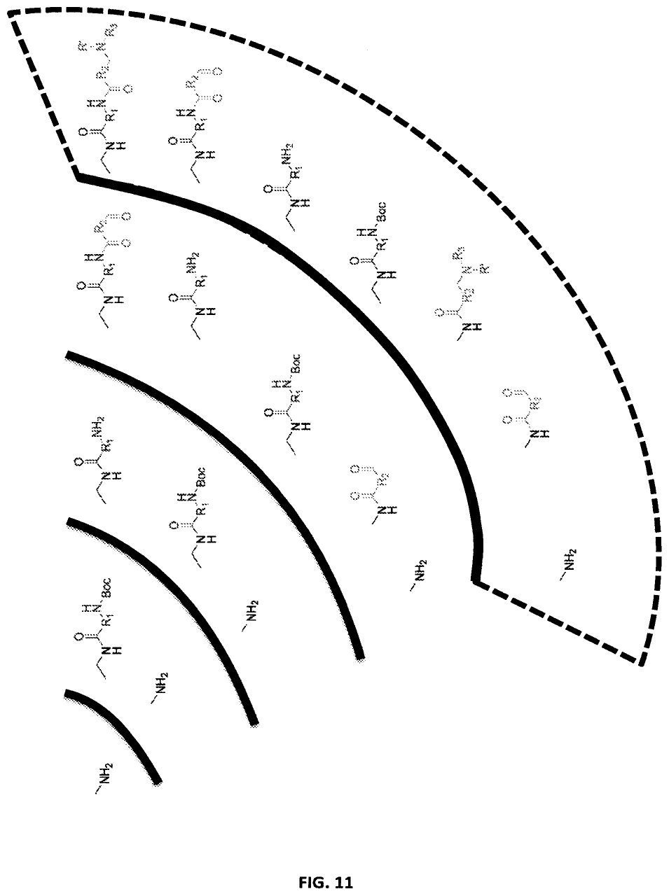

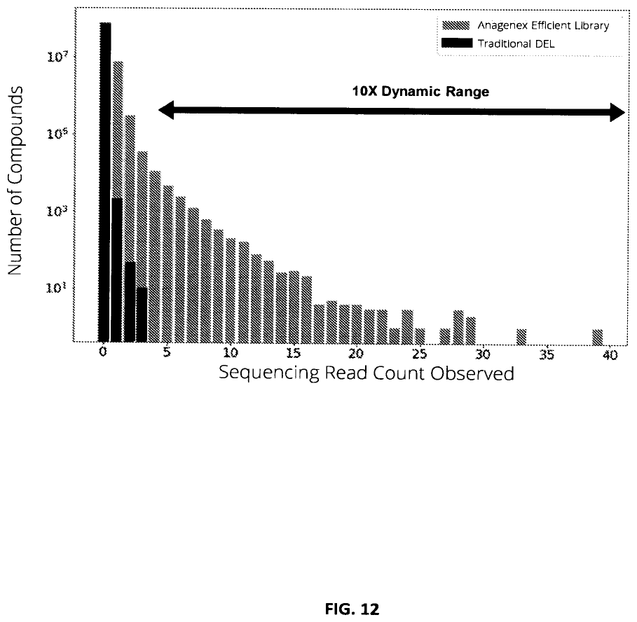

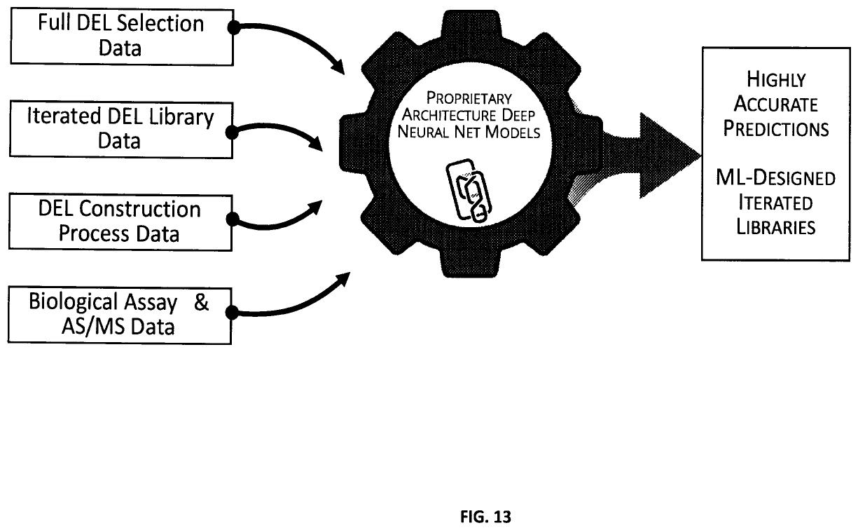

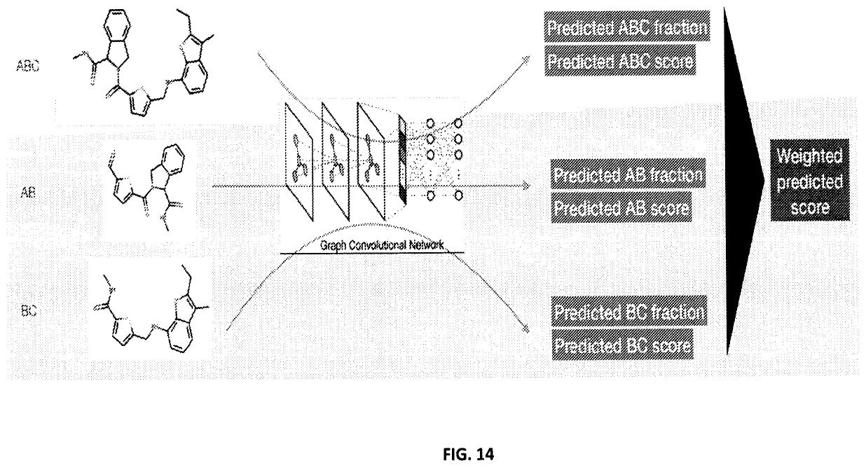

The platforms, systems, and methods disclosed herein may utilize a unique machine learning architecture specially tailored for DEL analysis. During DEL synthesis, individual building blocks are assembled in a combinatorial manner to generate millions or even billions of possible compounds. However, these chemical synthesis reactions are not necessarily 100% efficient, which means that intermediate and side products may be generated during DEL creation. As shown in FIG. 11, with each successive synthetic step adding another building block, more possible products are generated, including unreacted intermediate products and side products. The predicted compounds generated at the end of the assembly process may be modeled. In some embodiments of the platforms, systems, and methods disclosed herein, the model not just the end product compound but also the intermediate compounds are generated during intermediate synthetic steps. The conventional approach to ML on DEL does not account for these intermediate or side products. By contrast, embodiments of the platforms, systems, and methods disclosed herein utilize a neural network that incorporates the possible intermediate or side products in its training data. The result is that potential intermediate and side reactants or products present in the DEL are explicitly accounted for as possible factors explaining the read counts. By contrast, conventional approaches that assume only the final product was generated assume the final product was synthesized at 100% efficiency when, in fact, many compounds may have been synthesized at less than 30%. Therefore, the conventional approach is learning on ‘incorrect’ data, and the model's performance is likely to suffer. Accordingly, the machine learning model is given input data for all the final products and intermediate compounds. The ML model architecture described herein is designed to consider the final product and any intermediates as a group, and may further enhance that consideration with additional data in the form of measured chemical yields. This type of data is not present in the core DEL selection data which is the only data previously described model architectures can consider due to inherent limitations in their design. Because the ML model described herein may incorporate this additional information and is designed to consider the complete set of synthesized compounds along with the DEL selection data, it more accurately considers the underlying biochemical processes from the experiment. By leveraging that architecture and previously inaccessible information, it is able to dramatically improve its predictive accuracy.

In some embodiments, one or more quality control steps are carried out to control for reaction inefficiency. Measurements may be collected during one or more of the library construction (e.g., a DEL construction) steps. The measurements may include reaction efficiency, for example, mass spectrometry analysis of reaction products to identify relative abundance of the final product versus intermediate products or leftover reactants. Such information may be used to predict the fraction of each type of product or intermediate compound, which may be utilized to generate a weighting for the machine learning model to improve learning. In some embodiments, the measurements may be a compound descriptor. In some embodiments, the measurements are used to determine a compound descriptor and/or a fitness score. In some embodiments, a compound fitness score is in relation to an oral drug solubility, intestinal absorption, a permeability, a hERG toxicity, a CYP inhibition, a blood brain barrier permeability, a P-glycoprotein activity, plasma protein binding, and/or a binding affinity of

In some embodiments, the one DNA barcode or tag may represent all of the compounds (final and intermediate) in a given fraction. In some embodiments, the one DNA barcode or tag may be a compound descriptor. As described herein, since the reactions in a DEL may not be complete, a DNA tag will be affixed to several molecules (the final product, but also the intermediate products, the side products, etc.) that are produced during a series of predetermined synthesis steps. Therefore, the DNA tag may be understood as an indication that these synthesis steps were followed, not that the full molecule was generated. As a consequence, a unique DNA tag serves as an indicator that the molecule affixed to it is one of the molecules that can be produced in such a reaction scheme.

The data collected for a DEL experiment is used to train the machine learning model. A given data set can include a descriptor for each compound in the data set (compound descriptor), which may include a representation of the compound's molecular structure, the sequence of the compound's associated DNA tag, and the sequence read counts for the DNA tag. The data set may include additional information (e.g., descriptor) such as chemical properties or parameters (e.g., molecular weight). The data set may include a fitness score for each of the compounds in the data set In some embodiments a fitness score may be a score that is indicative of a compound having a desired property (e.g., binding affinity or activity in a biochemical assay or some ADME measurement). The trained model can then be used to evaluate another set of compounds to identify compounds predicted to have a desired property (e.g., high binding affinity to a target protein). The compounds may be identified by a fitness score of each compound. The new set of compounds predicted to have the desired property can be generated as a new or evolved DEL that is then subjected to another round of selection (e.g., binding assay and sequencing). The resulting data can be again used to further train and improve the machine learning model, which can be then used again to identify a new evolved DEL. This process can repeat iteratively a number of times until a smaller set of candidate compounds have been selected for further evaluation.

In some embodiments, the machine learning model is given input data generated from the DEL binding assay. The input data can be evaluated to determine an indication of binding such as a binding score. In some cases, the indication of binding can be used to categorize the given compound as a binder or non-binder with respect to a target protein (e.g., via a fitness score). In some embodiments, binding data maybe combined with a biochemical activity dataset to generate a fitness score. In some embodiments, the fitness score may be generated by analyzing the binding data and or the biochemical activity data to determine which compounds are more likely to have a desired biological effect.

In some embodiments, a fitness score may be the read count of a DEL compound and/or a number derived or calculated from the read counts of a single DNA compound. A non-limiting example of a fitness score derived or calculated from the read counts of a single DNA compound may be a ratio between the number of counts for that compound in a desired condition (the target condition) relative to the number of counts in one or more controls (non-target conditions). In some embodiments a derived or calculated fitness score may be a function of the read count and external information, such as compound synthesis data or data measuring the baseline abundance of compounds in the original library. Many different mathematical functions combining the read count with other data can be calculated to yield a fitness score. In some embodiments, a fitness score is in relation to an oral drug solubility, intestinal absorption, a permeability, a hERG toxicity, a CYP inhibition, a blood brain barrier permeability, a P-glycoprotein activity, plasma protein binding, and/or a binding affinity. In some embodiments, a fitness score is derived from a read count.

In some embodiments, a compound has a molecular structure that must be converted into a molecular representation suitable for input into a machine learning model. Examples of molecular representations include molecular graph representation (mapping the atoms and bonds of the molecular into nodes and edges), electrostatic representation, text representations such as SMILES or SELFIES, and geometric representation.

While graph representations are 2D data structures that do not explicitly recite spatial relationships between elements, 3D information can be incorporated into a graph representation. For example, a node feature matrix may include this information as node information (e.g., stereochemistry of an atom represented by the node), and the edge feature matrix may include this information as edge information (e.g., bond type).

In some embodiments, the DNA tag and sequence counts are processed from raw data into a suitable data set for machine learning analysis. In some cases, the sequence read counts are corrected upwards or downwards.

By contrast, traditional ML utilizes data and labels, for example, the molecular structure is the data (i.e., the structure that was assayed for binding to the target) and the label is the binding score (i.e., a score indicative of binding affinity generated based at least in part on sequence counts). In the event that the synthesis reaction steps did not have complete 100% efficiency, the data is one of several among a set of molecules and/or compounds. However, the methodology disclosed herein allows for machine learning analysis when it is unclear what data (molecular structure) corresponds to the label. The ML model architecture is designed to consider the final product and any intermediates as a group, and can further enhance that consideration with additional data in the form of measured chemical yields. This type of data is not present in the core DEL selection data which is the only data previously described model architectures can consider due to inherent limitations in their design. Because the ML model described herein can incorporate this additional information and is designed to consider the complete set of synthesized compounds along with the DEL selection data, it more accurately considers the underlying biochemical processes from the experiment. By leveraging that architecture and previously inaccessible information, it is able to dramatically improve its predictive accuracy.

In other words, the label or binding score is known, but there are multiple molecules within a set represented by the DNA code and as a result it is unclear what is the actual best binder in the set. With reference to FIG. 11, the top compound in the third synthesis step may be the best binder, but the other intermediate products may also experience some binding. While the DNA barcode cannot differentiate between the final product and the intermediate products due to incomplete synthesis, the model is given all the products corresponding to the DNA barcode in order to understand or learn from the set of compounds more fully. Accordingly, in some embodiments, the model sees the full molecule and not just disynthons as in some alternative methods. As a result, the model is configured based on the understanding that the DNA tag or barcode could represent any of the intermediate products (disynthons), reaction intermediates, or the full products (e.g., trisynthon).

In some embodiments, the machine learning model is used to generate predictions for new input data. For example, once a machine learning model has been trained on the input data generated from a DEL experiment, it will be able to receive new input data and generate predictions of fitness, for example, binding to a target protein. The prediction of fitness may be output as a fitness score. The fitness score may be a binding score or other properties. In some embodiments, the prediction comprises a composite score for multiple compound or drug properties. Non-limiting examples of other properties may include oral drug solubility, human intestinal absorption, permeability, hERG toxicity, CYP inhibition (2D6, 2C9), blood brain barrier permeability, P-glycoprotein activity, and plasma protein binding.

In some embodiments, the machine learning model learns one or more factors per possible molecule. The machine learning model, once trained, can then generate read count predictions by aggregating all the factors which can include one or more of: the matrix binding propensity of each of the set of possible molecules, the promiscuity propensity of the set of possible molecules, and the target binding propensity of the set of possible molecules.

To score molecules, the prediction may consist of the ‘target propensity’ of the target molecule (thus factoring out all other factors that are not of interest).

Alternatively, in some cases, a multiparameter score is generated that includes other properties instead of just a single one such as target propensity.

### Non-Limiting Numbered Embodiments

1. A method comprising: i) receiving a first input data set comprising first binding interaction information (e.g., compound descriptor) between a target molecule and a library (e.g., set) of compounds; ii) processing the first input data set using a machine learning module to generate a model representation of binding interactions (e.g., predictive compound descriptors), wherein the model is configured to predicting a binding fitness score between the target molecule and an input compound; iii) determining an updated library of compounds using the model representation of binding interactions, wherein the updated library of compounds comprises one or more new compounds predicted to bind the target molecule; iv) receiving a second input data set comprising second binding interaction information between the target molecule and the updated library of compounds; and v) processing the second input data set using the machine learning module to update the model representation of binding interactions, wherein at least the predictive accuracy of the updated model representation is improved.
2. The method of embodiment 1, the library of compounds is a combinatorial library of compounds.
3. The method of embodiment 2, wherein the combinatorial library of compounds is a DNA-encoded library.
4. The method of embodiment 2, wherein the combinatorial library of compounds is generated using a split pool method.
5. The method of embodiment 2, wherein the first input data set and the second input data set comprise the DNA sequencing read count of a DNA barcode tagged to each compound in the combinatorial library of compounds and at least one mapped structure corresponding to each DNA barcode.
6. The method of embodiment 1, wherein the target molecule is a biomolecule.
7. The method of embodiment 6, wherein the biomolecule comprises a macromolecule.
8. The method of embodiment 7, wherein the macromolecule comprises a polysaccharide, a carbohydrate, a lipid, or a nucleic acid.
9. The method of embodiment 7, wherein the macromolecule comprises a protein.
10. The method of embodiment 1, wherein the library of compounds comprises small molecule compounds.
11. The method of embodiment 10, wherein the small molecule compounds have a molecular weight of no more than 1000 Daltons.
12. The method of embodiment 10, wherein the library of compounds consists of between 100,000 and 1,000,000,000 small molecule compounds.
13. The method of embodiment 10, wherein the updated library of compounds consists of more than 10,000 small molecule compounds.
14. The method of embodiment 1, wherein the model representation is configured to predict binding for a plurality of compound fractions (e.g., compound viewed as a set of full, intermediate, and/or side product data) for each combinatorial synthetic chemistry pathway used in generating the library of compounds.
15. The method of embodiment 14, wherein one or more of the plurality of compound fractions each comprises a plurality of compounds corresponding to at least one target product and at least one side product.
16. The method of embodiment 14, wherein the model representation is configured to model the product and any side product(s) of a compound fraction generated from a synthetic step.
17. The method of embodiment 14, wherein each of the plurality of compound fractions is encoded with a DNA barcode that corresponds to the compound(s) (e.g., the full, intermediate and/or side products) within the compound fraction
18. The method of embodiment 14, wherein the model representation is configured to generate a binding score (e.g., a fitness score) for each compound fraction and/or compound in each compound fraction.
19. The method of embodiment 1, wherein the model representation comprises a neural network.
20. The method of embodiment 19, wherein the neural network is a graph neural network.
21. The method of embodiment 1, wherein the method further comprise iteratively repeating steps (c)-(e) to improve the predictive accuracy of the model representation.
22. The method of embodiment 21, wherein steps (c)-(e) are iteratively repeated until at least 1, 10, 50, 100, 200, 300, 400, or 500 compounds are identified as having a predicted affinity for the target molecule above a minimum threshold.
23. The method of embodiment 1, wherein the method further comprises weighting the binding interactions of the model representation (e.g., predictive compound descriptors) based on experimental data corresponding to efficiency of synthetic steps used to generate the library of compounds, thereby reducing signal to noise.
24. The method of embodiment 23, wherein the experimental data comprises abundance information based on mass spectrometry analysis of the compound fraction comprising one or more synthetic compound products generated from each synthetic step.
25. The method of embodiment 1, wherein the model representation is a graph convolutional neural network configured to receive a graph representation of a given compound as input data.
26. The method of embodiment 25, wherein the graph representation of the given compound comprises a graph data structure composed of vertices and edges.
27. The method of embodiment 1, wherein the method further comprises conducting a binding affinity experiment between the target molecule and the library of compounds.
28. The method of embodiment 27, wherein the binding affinity experiment comprises incubating the target molecule with the library of compounds and purifying the target molecule together with any bound compounds.
29. The method of embodiment 27, wherein the binding affinity experiment comprises eluting the bound compounds, wherein the bound compounds are tagged with DNA barcodes.
30. The method of embodiment 28, wherein the method further comprises amplifying the DNA barcodes.
31. The method of embodiment 29, wherein the method further comprises sequencing the DNA barcodes to obtain read count data for the bound compounds tagged with the DNA barcodes.
32. The method of embodiment 29, wherein the sequencing comprises deep sequencing to obtain complete sequencing of the DNA barcodes.
33. The method of embodiment 31, wherein selection coverage of the compounds in the library of compounds is at least 80%, 85%, 90%, 95%, 99%, or 100%.
34. The method of embodiment 27, wherein the binding affinity experiment is performed for one or more iterative rounds of input data generation and determining the updated library of compounds using the model representation.
35. The method of embodiment 34, wherein the binding affinity experiment comprises calibrating the amount of input material to the number of rounds.
36. The method of embodiment 34, wherein the one or more iterative rounds comprises at least one, two, three, four, or five rounds of input data generation and determining the updated library of compounds using the model representation.
37. The method of embodiment 1, wherein the method further comprises conducting a binding affinity experiment between the target molecule and the updated library of compounds after step (c).
38. The method of embodiment 1, wherein the library of compounds is selected as a subset of a larger library of possible compounds, and wherein the updated library of compounds comprises the one or more new compounds identified from the larger library of possible compounds as potential binders of the target molecule.
39. The method of embodiment 1, wherein the method further comprises utilizing deep sequencing for complete sequencing and not partial reads. In some embodiments, the selection coverage of the molecules in the DEL is at least 50%, 55%, 60%, 65%, 70%, 75%, 80%, 85%, 90%, 95%, 99%, or 100%. In some embodiments, the selection coverage ranges from 50%, 55%, 60%, 65%, 70%, or 75% to 80%, 85%, 90%, 95%, 99%, or 100%.
40. A computer-implemented system comprising: a digital processing device comprising: at least one processor, an operating system configured to perform executable instructions, a memory, and a computer program including instructions executable by the digital processing device to perform the perform the method of any one of embodiments 1-39.
41. A non-transitory computer-readable storage media encoded with a computer program including instructions executable by one or more processors to perform the method of any one of embodiments 1-39.

### Terms and Definitions

Unless otherwise defined, all technical terms used herein have the same meaning as commonly understood by one of ordinary skill in the art to which this disclosure belongs.

As used herein, the singular forms “a,” “an,” and “the” include plural references unless the context clearly dictates otherwise. Any reference to “or” herein is intended to encompass “and/or” unless otherwise stated.

As used herein, the term “about” in some cases refers to an amount that is approximately the stated amount.

As used herein, the term “about” in some cases refers to an amount that is near the stated amount by 10%, 5%, or 1%, including increments therein.

As used herein, the term “about” in reference to a percentage in some cases refers to an amount that is greater or less the stated percentage by 10%, 5%, or 1%, including increments therein.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the term “building block(s)” may be any chemical structure that may combined or added to another chemical structure to build one or more compounds. In some embodiments, a compound may function as a building block wherein a compound may be combined or added to one or more building blocks to build a new compound. In some embodiments one or more compounds are used as building blocks to build new compounds in a new DEL. In a non-limiting example, a monosynthon may be a first building block and a second monosynthon may be a second building block, wherein the two building blocks react together to form a new disynthon compound. The disynthon may then be used as a building block along with one or more building blocks to build one or more trisynthons and/or polysynthons.

As used herein, the term “compound descriptor” refers to any data or information associated with a compound. The data or information may include but is not limited to a binding affinity of the compound to a target molecule (optionally wherein the target molecule is a drug target such as a protein or a nucleic acid), activity of the compound, a physical property of the compound (e.g., lipophilicity), toxicity of the compound, stability of the compound, permeability of the compound, sequencing reads associated with an abundance of the compound in an experiment, information related to synthesis of the compound, a structure of the compound, labeling data, process quality control data, or yield associated with synthesis of the compound, sequencing data, labeling data, product data, synthesis efficiency data, mass spectrometry data, compound fraction data, binding data, matrix binding data, promiscuity data, structure data, or building block validation data or a combination of two or more thereof. In some embodiments, a compound descriptor may be in the form of a graph, chart, number/value, label, text, data set, image, etc.

### Machine Learning

A machine learning model can comprise one or more of various machine learning models. In some embodiments, the machine learning model can comprise one machine learning model. In some embodiments, the machine learning model can comprise a plurality of machine learning models. In some embodiments, the machine learning model can comprise a neural network model. In some embodiments, the machine learning model can comprise a random forest model. In some embodiments, the machine learning model can comprise a manifold learning model. In some embodiments, the machine learning model can comprise a hyperparameter learning model. In some embodiments, the machine learning model can comprise an active learning model.

A graph, graph model, and graphical model can refer to a method of conceptualizing or organizing information into a graphical representation comprising nodes and edges. In some embodiments, a graph can refer to the principle of conceptualizing or organizing data, wherein the data may be stored in a various and alternative forms such as linked lists, dictionaries, spreadsheets, arrays, in permanent storage, in transient storage, and so on, and is not limited to specific embodiments disclosed herein. In some embodiments, the machine learning model can comprise a graph model.

The machine learning model can comprise a variety of manifold learning algorithms. In some embodiments, the machine learning model can comprise a manifold learning algorithm. In some embodiments, the manifold learning algorithm is principal component analysis. In some embodiments, the manifold learning algorithm is a uniform manifold approximation algorithm. In some embodiments, the manifold learning algorithm is an isomap algorithm. In some embodiments, the manifold learning algorithm is a locally linear embedding algorithm. In some embodiments, the manifold learning algorithm is a modified locally linear embedding algorithm. In some embodiments, the manifold learning algorithm is a Hessian eigenmapping algorithm. In some embodiments, the manifold learning algorithm is a spectral embedding algorithm. In some embodiments, the manifold learning algorithm is a local tangent space alignment algorithm. In some embodiments, the manifold learning algorithm is a multi-dimensional scaling algorithm. In some embodiments, the manifold learning algorithm is a t-distributed stochastic neighbor embedding algorithm (t-SNE). In some embodiments, the manifold learning algorithm is a Barnes-Hut t-SNE algorithm.

The terms reducing, dimensionality reduction, projection, component analysis, feature space reduction, latent space engineering, feature space engineering, representation engineering, or latent space embedding can refer to a method of transforming a given input data with an initial number of dimensions to another form of data that has fewer dimensions than the initial number of dimensions. In some embodiments, the terms can refer to the principle of reducing a set of input dimensions to a smaller set of output dimensions.

The term normalizing can refer to a collection of methods for adjusting a dataset to align the dataset to a common scale. In some embodiments, a normalizing method can comprise multiplying a portion or the entirety of a dataset by a factor. In some embodiments, a normalizing method can comprise adding or subtracting a constant from a portion or the entirety of a dataset. In some embodiments, a normalizing method can comprise adjusting a portion or the entirety of a dataset to a known statistical distribution. In some embodiments, a normalizing method can comprise adjusting a portion or the entirety of a dataset to a normal distribution. In some embodiments, a normalizing method can comprise adjusting the dataset so that the signal strength of a portion or the entirety of a dataset is about the same.

Converting can comprise one or more steps of various of conversions of data. In some embodiments, converting can comprise normalizing data. In some embodiments, converting can comprise performing a mathematical operation that computes a score based on a distance between 2 points in the data. In some embodiments, the distance can comprise a distance between two edges in a graph. In some embodiments, the distance can comprise a distance between two nodes in a graph. In some embodiments, the distance can comprise a distance between a node and an edge in a graph. In some embodiments, the distance can comprise a Euclidean distance. In some embodiments, the distance can comprise a non-Euclidean distance. In some embodiments, the distance can be computed in a frequency space. In some embodiments, the distance can be computed in Fourier space. In some embodiments, the distance can be computed in Laplacian space. In some embodiments, the distance can be computed in spectral space. In some embodiments, the mathematical operation can be a monotonic function based on the distance. In some embodiments, the mathematical operation can be a non-monotonic function based on the distance. In some embodiments, the mathematical operation can be an exponential decay function. In some embodiments, the mathematical operation can be a learned function.

In some embodiments, converting can comprise transforming a data in one representation to another representation. In some embodiments, converting can comprise transforming data into another form of data with less dimensions. In some embodiments, converting can comprise linearizing one or more curved paths in the data. In some embodiments, converting can be performed on data comprising data in Euclidean space. In some embodiments, converting can be performed on data comprising data in graph space. In some embodiments, converting can be performed on data in a discrete space. In some embodiments, converting can be performed on data comprising data in frequency space. In some embodiments, converting can transform data in discrete space to continuous space, continuous space to discrete space, graph space to continuous space, continuous space to graph space, graph space to discrete space, discrete space to graph space, or any combination thereof. In some embodiments, converting can comprise transforming data in discrete space into a frequency domain. In some embodiments, converting can comprise transforming data in continuous space into a frequency domain. In some embodiments, converting can comprise transforming data in graph space into a frequency domain.

In some embodiments, the methods of the disclosure further comprise reducing compound descriptors to a reduced descriptor space using a machine learning model. In some embodiments, the method further comprises clustering the reduced descriptor space to determine one or more groups of compound descriptors with similar features.

In some embodiments, reducing can comprise transforming a given input data with any initial number of dimensions to another form of data that has any number of dimensions fewer than the initial number of dimensions. In some embodiments, reducing can comprise transforming input data into another form of data with fewer dimensions. In some embodiments, reducing can comprise linearizing one or more curved paths in the input data to the output data. In some embodiments, reducing can be performed on data comprising data in Euclidean space. In some embodiments, reducing can be performed on data comprising data in graph space. In some embodiments, reducing can be performed on data in a discrete space. In some embodiments, reducing can transform data in discrete space to continuous space, continuous space to discrete space, graph space to continuous space, continuous space to graph space, graph space to discrete space, discrete space to graph space, or any combination thereof.

The terms clustering, cluster analysis, or generating modules can refer to a method of grouping samples in a dataset by some measure of similarity. Samples can be grouped in a set space, for example, element ‘a’ is in set ‘A’. Samples can be grouped in a continuous space, for example, element ‘a’ is a point in Euclidean space with distance ‘l’ away from the centroid of elements comprising cluster ‘A’. Samples can be grouped in a graph space, for example, element ‘a’ is highly connected to elements comprising cluster ‘A’. These terms can refer to the principle of organizing a plurality of elements into groups in some mathematical space based on some measure of similarity.

Clustering can comprise grouping any number of samples in a dataset by any quantitative measure of similarity. In some embodiments, clustering can comprise K-means clustering. In some embodiments, clustering can comprise hierarchical clustering. In some embodiments, clustering can comprise using random forest models. In some embodiments, clustering can comprise boosted tree models. In some embodiments, clustering can comprise using support vector machines. In some embodiments, clustering can comprise calculating one or more N−1 dimensional surfaces in N-dimensional space that partitions a dataset into clusters. In some embodiments, clustering can comprise distribution-based clustering. In some embodiments, clustering can comprise fitting a plurality of prior distributions over the data distributed in N-dimensional space. In some embodiments, clustering can comprise using density-based clustering. In some embodiments, clustering can comprise using fuzzy clustering. In some embodiments, clustering can comprise computing probability values of a data point belonging to a cluster. In some embodiments, clustering can comprise using constraints. In some embodiments, clustering can comprise using supervised learning. In some embodiments, clustering can comprise using unsupervised learning.

In some embodiments, clustering can comprise grouping samples based on similarity. In some embodiments, clustering can comprise grouping samples based on quantitative similarity. In some embodiments, clustering can comprise grouping samples based on one or more features of each sample. In some embodiments, clustering can comprise grouping samples based on one or more labels of each sample. In some embodiments, clustering can comprise grouping samples based on Euclidean coordinates. In some embodiments, clustering can comprise grouping samples based the features of the nodes and edges of each sample.

In some embodiments, comparing can comprise comparing between a first group and different second group. In some embodiments, a first or a second group can each independently be a cluster. In some embodiments, a first or a second group can each independently be a group of clusters. In some embodiments, comparing can comprise comparing between one cluster with a group of clusters. In some embodiments, comparing can comprise comparing between a first group of clusters with second group of clusters different than the first group. In some embodiments, one group can be one sample. In some embodiments, one group can be a group of samples. In some embodiments, comparing can comprise comparing between one sample versus a group of samples. In some embodiments, comparing can comprise comparing between a group of samples versus a group of samples.

### Neural Network

In some embodiments, systems and methods of the present disclosure may comprise or comprise using a neural network. The neural network may comprise various architectures, loss functions, optimization algorithms, assumptions, and various other neural network design choices. In some embodiments, the neural network comprises an encoder. In some embodiments, the neural network comprises a decoder. In some embodiments, the neural network comprises a bottleneck architecture comprising the encoder and the decoder. In some embodiments, the bottleneck architecture comprises an autoencoder. In some embodiments, the neural network comprises a language model. In some embodiments, the neural network comprises a transformer model.

Various types of layers may be used a neural network. In some embodiments, the neural network comprises a convolutional layer. In some embodiments, the neural network comprises a densely connected layer. In some embodiments, the neural network comprises a skip connection. In some embodiments, the neural network may comprise graph convolutional layers. In some embodiments, the neural network may comprise message passing layers. In some embodiments, the neural network may comprise attention layers. In some embodiments, the neural network may comprise recurrent layers. In some embodiments, the neural network may comprise a gated recurrent unit. In some embodiments, the neural network may comprise reversible layers. In some embodiments, the neural network may comprise a neural network with a bottleneck layer. In some embodiments, the neural network may comprise residual blocks. In some embodiments, the neural network may comprise one or more dropout layers. In some embodiments, the neural network may comprise one or more locally connected layers. In some embodiments, the neural network may comprise one or more batch normalization layers. In some embodiments, the neural network may comprise one or more pooling layers. In some embodiments, the neural network may comprise one or more upsampling layers. In some embodiments, the neural network may comprise one or more max-pooling layers.

In some embodiments, the neural network comprises a graph model. In some embodiments, a graph, graph model, and graphical model can refer to a method that models data in a graphical representation comprising nodes and edges. In some embodiments, the data may be stored in a various and alternative forms such as linked lists, dictionaries, spreadsheets, arrays, in permanent storage, in transient storage, and so on, and is not limited to specific embodiments disclosed herein.

In some embodiments, the neural network may comprise an autoencoder. In some embodiments, the neural network may comprise a variational autoencoder. In some embodiments, the neural network may comprise a generative adversarial network. In some embodiments, the neural network may comprise a flow model. In some embodiments, the neural network may comprise an autoregressive model.

The neural network may comprise various activation functions. In some embodiments, an activation function may be a non-linearity. In some embodiments, the neural network may comprise one or more activation functions. In some embodiments, the neural network may comprise a ReLU, softmax, tanh, sigmoid, softplus, softsign, selu, elu, exponential, LeakyReLU, or any combination thereof. Various activation functions may be used with a neural network, without departing from the inventive concepts disclosed herein.

### Training

Various loss functions can be used to train the neural network. In some embodiments, the neural network may comprise a regression loss function. In some embodiments, the neural network may comprise a logistic loss function. In some embodiments, the neural network may comprise a variational loss. In some embodiments, the neural network may comprise a prior. In some embodiments, the neural network may comprise a Gaussian prior. In some embodiments, the neural network may comprise a non-Gaussian prior. In some embodiments, the neural network may comprise a Laplacian prior. In some embodiments, the neural network may comprise a Gaussian posterior. In some embodiments, the neural network may comprise a non-Gaussian posterior. In some embodiments, the neural network may comprise a Laplacian posterior. In some embodiments, the neural network may comprise an adversarial loss. In some embodiments, the neural network may comprise a reconstruction loss. In some embodiments, the loss functions may be formulated to optimize a regression loss, an evidence-based lower bound, a maximum likelihood, Kullback-Leibler divergence, applied with various distribution functions such as Gaussians, non-Gaussian, mixtures of Gaussians, mixtures of logistic functions, and so on.

Various optimizers can be used to train the neural network. In some embodiments, the neural network may be trained with the Adam optimizer. In some embodiments, the neural network may be trained with the stochastic gradient descent optimizer. In some embodiments, the neural network may be trained with an active learning algorithm. A neural network may be trained with various loss functions whose derivatives may be computed to update one or more parameters of the neural network. A neural network may be trained with hyperparameter searching algorithms. In some embodiments, the neural network hyperparameters are optimized with Gaussian Processes.

Various training protocols can be used while training the neural network. In some embodiments, the neural network may be trained with train/validation/test data splits. In some embodiments, the neural network may be trained with k-fold data splits, with any positive integer for k.

Training the neural network can involve providing inputs to the untrained neural network to generate predicted outputs, comparing the predicted outputs to the expected outputs, and updating the neural network's parameters to account for the difference between the predicted outputs and the expected outputs. Based on the calculated difference, a gradient with respect to each parameter may be calculated by backpropagation to update the parameters of the neural network so that the output value(s) that the neural network computes are consistent with the examples included in the training set. This process may be iterated for a certain number of iterations or until some stopping criterion is met.

### Computing System

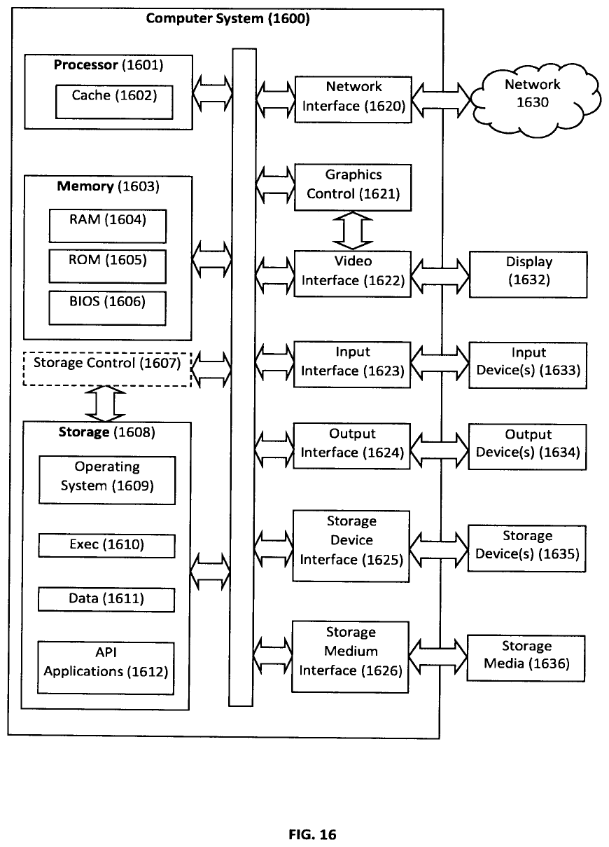

Referring to FIG. 16, a block diagram is shown depicting an exemplary machine that includes a computer system 1600 (e.g., a processing or computing system) within which a set of instructions can execute for causing a device to perform or execute any one or more of the aspects and/or methodologies for static code scheduling of the present disclosure. The components in FIG. 16 are examples only and do not limit the scope of use or functionality of any hardware, software, embedded logic component, or a combination of two or more such components implementing particular embodiments.

Computer system 1600 may include one or more processors 1601, a memory 1603, and a storage 1608 that communicate with each other, and with other components, via a bus 1640. The bus 1640 may also link a display 1632, one or more input devices 1633 (which may, for example, include a keypad, a keyboard, a mouse, a stylus, etc.), one or more output devices 1634, one or more storage devices 1635, and various tangible storage media 1636. All of these elements may interface directly or via one or more interfaces or adaptors to the bus 1640. For instance, the various tangible storage media 1636 can interface with the bus 1640 via storage medium interface 1626. Computer system 1600 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 1600 includes one or more processor(s) 1601 (e.g., central processing units (CPUs) or general purpose graphics processing units (GPGPUs)) that carry out functions. Processor(s) 1601 optionally contains a cache memory unit 1602 for temporary local storage of instructions, data, or computer addresses. Processor(s) 1601 are configured to assist in execution of computer readable instructions. Computer system 1600 may provide functionality for the components depicted in FIG. 16 as a result of the processor(s) 1601 executing non-transitory, processor-executable instructions embodied in one or more tangible computer-readable storage media, such as memory 1603, storage 1608, storage devices 1635, and/or storage medium 1636. The computer-readable media may store software that implements particular embodiments, and processor(s) 1601 may execute the software. Memory 1603 may read the software from one or more other computer-readable media (such as mass storage device(s) 1635, 1636) or from one or more other sources through a suitable interface, such as network interface 1620. The software may cause processor(s) 1601 to carry out one or more processes or one or more steps of one or more processes described or illustrated herein. Carrying out such processes or steps may include defining data structures stored in memory 1603 and modifying the data structures as directed by the software.

The memory 1603 may include various components (e.g., machine readable media) including, but not limited to, a random access memory component (e.g., RAM 1604) (e.g., static RAM (SRAM), dynamic RAM (DRAM), ferroelectric random access memory (FRAM), phase-change random access memory (PRAM), etc.), a read-only memory component (e.g., ROM 1605), and any combinations thereof. ROM 1605 may act to communicate data and instructions unidirectionally to processor(s) 1601, and RAM 1604 may act to communicate data and instructions bidirectionally with processor(s) 1601. ROM 1605 and RAM 1604 may include any suitable tangible computer-readable media described below. In one example, a basic input/output system 1606 (BIOS), including basic routines that help to transfer information between elements within computer system 1600, such as during start-up, may be stored in the memory 1603.

Fixed storage 1608 is connected bidirectionally to processor(s) 1601, optionally through storage control unit 1607. Fixed storage 1608 provides additional data storage capacity and may also include any suitable tangible computer-readable media described herein. Storage 1608 may be used to store operating system 1609, executable(s) 1610, data 1611, applications 1612 (application programs), and the like. Storage 1608 can also include an optical disk drive, a solid-state memory device (e.g., flash-based systems), or a combination of any of the above. Information in storage 1608 may, in appropriate cases, be incorporated as virtual memory in memory 1603.

In one example, storage device(s) 1635 may be removably interfaced with computer system 1600 (e.g., via an external port connector (not shown)) via a storage device interface 1625. Particularly, storage device(s) 1635 and an associated machine-readable medium may provide non-volatile and/or volatile storage of machine-readable instructions, data structures, program modules, and/or other data for the computer system 1600. In one example, software may reside, completely or partially, within a machine-readable medium on storage device(s) 1635. In another example, software may reside, completely or partially, within processor(s) 1601.

Bus 1640 connects a wide variety of subsystems. Herein, reference to a bus may encompass one or more digital signal lines serving a common function, where appropriate. Bus 1640 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 1600 may also include an input device 1633. In one example, a user of computer system 1600 may enter commands and/or other information into computer system 1600 via input device(s) 1633. Examples of an input device(s) 1633 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 1633 may be interfaced to bus 1640 via any of a variety of input interfaces 1623 (e.g., input interface 1623) including, but not limited to, serial, parallel, game port, USB, FIREWIRE, THUNDERBOLT, or any combination of the above.

In particular embodiments, when computer system 1600 is connected to network 1630, computer system 1600 may communicate with other devices, specifically mobile devices and enterprise systems, distributed computing systems, cloud storage systems, cloud computing systems, and the like, connected to network 1630. Communications to and from computer system 1600 may be sent through network interface 1620. For example, network interface 1620 may receive incoming communications (such as requests or responses from other devices) in the form of one or more packets (such as Internet Protocol (IP) packets) from network 1630, and computer system 1600 may store the incoming communications in memory 1603 for processing. Computer system 1600 may similarly store outgoing communications (such as requests or responses to other devices) in the form of one or more packets in memory 1603 and communicated to network 1630 from network interface 1620. Processor(s) 1601 may access these communication packets stored in memory 1603 for processing.

Examples of the network interface 1620 include, but are not limited to, a network interface card, a modem, and any combination thereof. Examples of a network 1630 or network segment 1630 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 1630, may employ a wired and/or a wireless mode of communication. In general, any network topology may be used.

Information and data can be displayed through a display 1632. Examples of a display 1632 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 1632 can interface to the processor(s) 1601, memory 1603, and fixed storage 1608, as well as other devices, such as input device(s) 1633, via the bus 1640. The display 1632 is linked to the bus 1640 via a video interface 1622, and transport of data between the display 1632 and the bus 1640 can be controlled via the graphics control 1621. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 1632, computer system 1600 may include one or more other peripheral output devices 1634 including, but not limited to, an audio speaker, a printer, a storage device, and any combinations thereof. Such peripheral output devices may be connected to the bus 1640 via an output interface 1624. Examples of an output interface 1624 include, but are not limited to, a serial port, a parallel connection, a USB port, a FIREWIRE port, a THUNDERBOLT port, and any combinations thereof.

In addition or as an alternative, computer system 1600 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

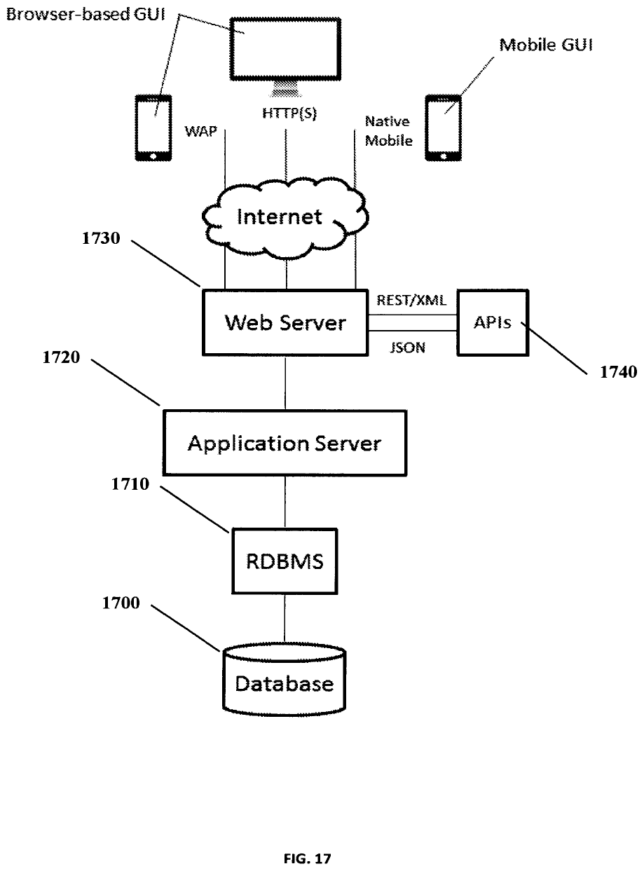

Referring to FIG. 17, in a particular embodiment, an application provision system comprises one or more databases 1700 accessed by a relational database management system (RDBMS) 1710. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 1720 (such as Java servers, .NET servers, PHP servers, and the like) and one or more web servers 1730 (such as Apache, IIS, GWS and the like). The web server(s) optionally expose one or more web services via app application programming interfaces (APIs) 1740. Via a network, such as the Internet, the system provides browser-based and/or mobile native user interfaces.

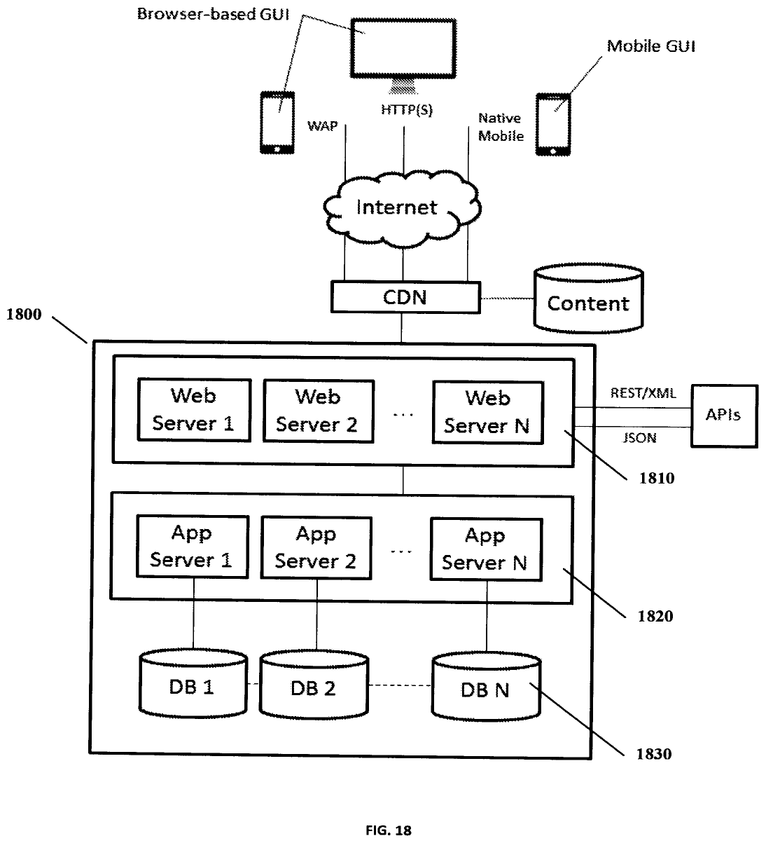

Referring to FIG. 18, in a particular embodiment, an application provision system alternatively has a distributed, cloud-based architecture 1800 and comprises elastically load balanced, auto-scaling web server resources 1810 and application server resources 1820 as well synchronously replicated databases 1830.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, and methods disclosed herein include one or more databases, or use of the same. In view of the disclosure provided herein, those of skill in the art will recognize that many databases are suitable for storage and retrieval of DEL information and associated experimental data collected for one or more conditions. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### EXAMPLES

The following illustrative examples are representative of embodiments of the software applications, systems, and methods described herein and are not meant to be limiting in any way.

### Example 1

An example iterative machine learning-based DEL platform disclosed herein was used to identify compounds that bind a target that is known to be challenging for DEL. This target binds DNA, and because DELs contain a lot of DNA, the target will create a lot of noise in the selection. In addition, in order to be successful against this target, a compound must disrupt a protein-protein interaction (PPI). PPIs are known to be challenging for small molecules, although this particular compound is known and has gone to clinical trials, so the problem is very difficult but not impossible.

An initial DEL composed of approximately 450 million compounds was assessed in a binding assay against the target. A total of approximately 3 conditions were tested, including [conditions 1, 2, 3, control, etc.]. The sequencing results (hits) were then input into a GCNN model to identify an evolved DEL composed of 350,000 compounds. This step was iterated 1 time until the top binding compounds were identified that were predicted to bind to the target. These 135 compounds identified by the ML-enabled engine were tested and compared to the known clinical compound as shown in Table 1.

TABLE 1
Affinity Comparison Between Clinical Compound
(control) and Predicted Compounds

| Compound | Affinity/Activity | Molecular Weight | cLogP |
|---|---|---|---|
| Clinical Compound | 4 nM IC50 | 532.1 | 8.23 |
| AGX-59 | 4.5x better Kd | 321.3 | 2.62 |
| AGX-87 | 4.3x better Kd | 313.4 | 2.8 |
| AGX-47 | 4.2x better Kd | 318.3 | 3.05 |
| AGX-54 | 1.3x better Kd | 310.4 | 1.88 |
| AGX-39 | Equivalent Kd | 359.4 | 1.04 |
| AGX-46 | Equivalent Kd | 354.4 | 1.28 |

Of the 135 compounds we predicted might work, 21% were confirmed to bind the target, of which several matched or exceeded the clinical compound's performance in a binding assay (affinity/activity) as shown in Table 1. Two thirds of these confirmed compounds were also experimentally determined to disrupt the relevant PPI. A compound that performs well in an assay is useless if it lacks drug-like properties. As shown in Table 1, nearly all of the identified compounds fall well within the drug-like space (see above table for appropriate molecular weights and c Log P, which are even better than the clinical compound).

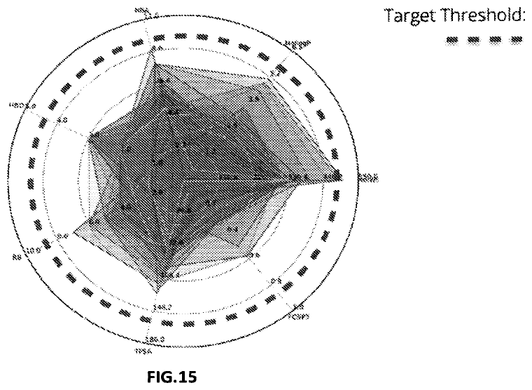

In addition, FIG. 15 provides a star chart plotting c Log P, Molecular Weight (MW), Hydrogen Bond Acceptors (HBA), Hydrogen Bond Donors (HBD), Rotatable Bonds (RB), Topological Polar Surface Area (TPSA), and SP3 Fraction (fSP3). Good candidate drugs will stay within the next-to-last circle on the plot. Almost all compounds fall well within this boundary, with a small number being a little heavier, touching roughly 500 MW. Accordingly, the iterative ML-driven DEL discovery process was successfully validated against a difficult target with higher biochemical performance compared to a known clinical compound that advanced to clinical trials.

ML models often perform well on the data they were trained on but fail to perform well outside that dataset. However, the instant ML model performed well outside of the bounds of the DEL the ML was trained on. Specifically, the training set was plotted in yellow, the predicted (and also successful) molecules in red, and the newly evolved DEL library in grey. To a first approximation, on this plot, closely related compounds will group themselves more closely together than less closely related compounds. As shown in FIG. 15, the red dots are generally outside the yellow area, showing that our ML models can identify high performing compounds outside of the training set. Because the ML model generalized outside the training set, they can be useful when a medicinal chemist proposes a molecule that looks different from what has been built in the past. Therefore, these models can guide the downstream compound optimization process.

### Example 2

Based on the methods of Example 1, an evolved library was built based on the results from the initial selections. The compounds were selected against the same target, and the model was updated with the resulting data. Finally, the newly updated model was used to answer two questions:

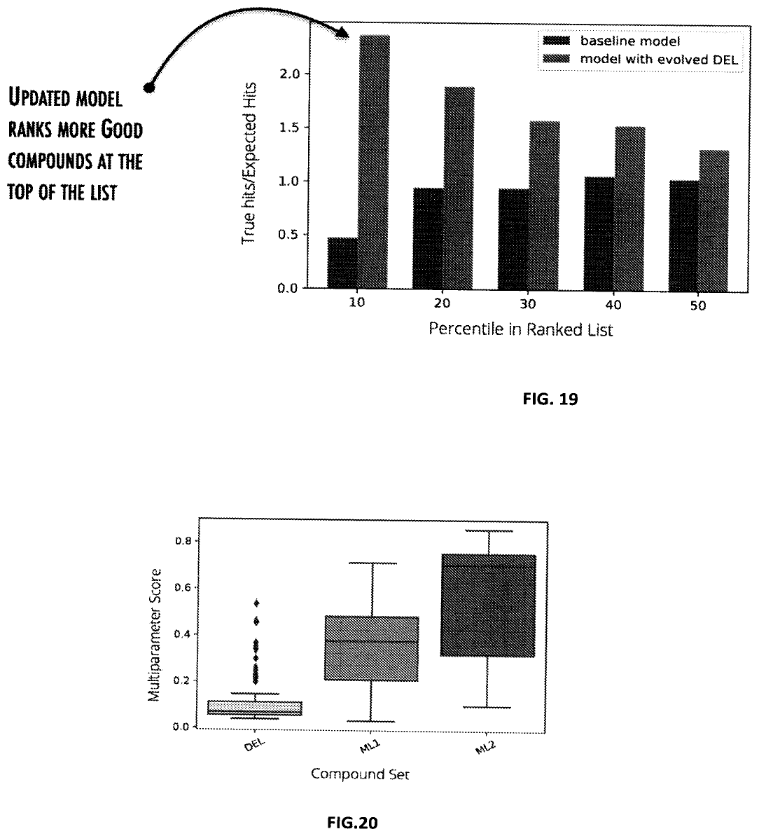

1) Given the initial set of tested compounds, determine the ranking of the confirmed compounds. The model generated score predictions for the compounds, which were used to determine the ranking. As shown in FIG. 19, the plot shows the top of the list is now enriched with more true positives than expected. In other words, more compounds are ranked closer to the top of the list and this model performs better due to having seen an evolved DEL library.

2) Predict a new set of compounds to test now that the model have a better understanding of what compounds should work. Specifically, the question was whether compounds could be found within the new set of compounds that have better predicted properties for later stage drug discovery such as ADME/Toxicity.

To measure this, a composite score was created for compounds including the following predicted drug properties: oral drug solubility, human intestinal absorption, permeability, hERG toxicity, CYP inhibition (2D6, 2C9), blood brain barrier permeability, P-glycoprotein activity, and plasma protein binding. On this metric, a score of 1 is perfect, and compounds with a lower score tend to have a worse set of predicted properties The relative importance of each factor can be customized for each target as needed, and either experimental measurements or predicted values can be used. Next, the values for compounds within the initial DEL, the first set of predicted compounds (ML1), and the second set of predicted compounds (ML2) were plotted. As shown in FIG. 20, the compound properties improved as the compounds progressed from pure DEL compounds to ML predicted compounds (ML1). They improved further when the model was shown more data (i.e., updated) and asked to predict yet another set of compounds (ML2). These results show the value of iterative ML-driven evolved DEL libraries in improving model performance and helping find better drugs faster.

### Example 3

The high-signal to noise, massively parallel datasets enabled by the present disclosure allow compound specificity questions to be evaluated in an unprecedented manner. Specifically, the platforms, systems, and methods disclosed herein allows for comparison, at scale, of several different isoforms of a protein or domain and help identify compounds that hit one isoform and none of the others. To demonstrate this capability, two experiments were conducted, and the results plotted to show how this platform is capable of identifying highly specific compounds.

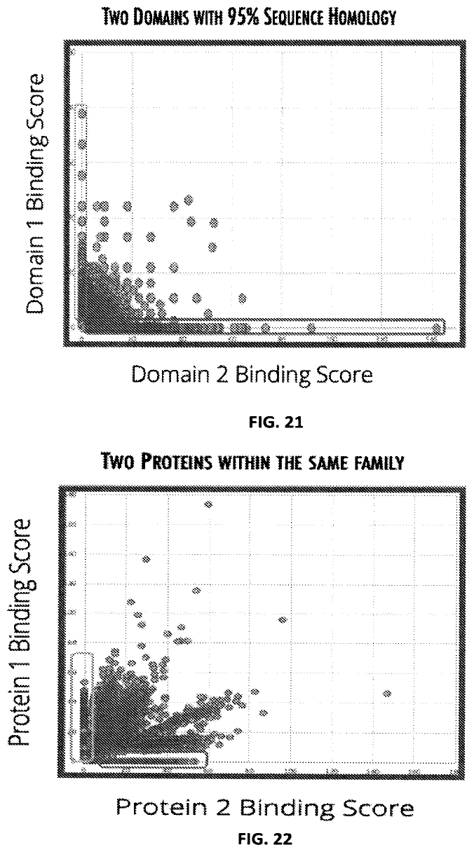

Two highly related protein domains (95% sequence similarity) within a single protein were evaluated. Each compound is represented by a blue dot. As shown in FIG. 21, the compound score for Domain 1 was plotted on the Y axis, and the compound score for Domain 2 was plotted on the X axis. A compound that is specific for Domain 1 will show up only on the Y axis, and a better compound will have a larger score. Similarly, a compound specific for Domain 2 shows up only on the X axis. In this example, a large number of compounds were identified that were specific for each of the two highly similar domains but not the other.

This experiment was repeated using two proteins within the same class of chromatin regulators. The results shown in FIG. 22 indicate a large number of molecules that are predicted to specifically bind only one of the two related proteins. This experiment further demonstrates that the present platform is capable of identifying a large number of potentially specific molecules for two closely related proteins where traditional drug discovery struggles to identify specific compounds.

### Example 4

The method described herein demonstrates a method of training a custom GNN to develop a predictive model that incorporates intermediate product data in the probabilistic modeling of counts, wherein the trained model predicts the enrichments of the full product and the enrichments of intermediate products.

In this case, a first data set was built, by making an initial DEL from a combination of 15 proprietary DEL, totaling 700 million unique molecules. The initial DEL may be built form one or a combination of any number of DEL libraries (e.g., from million to billions of unique molecules). The initial DEL library then sequenced, producing 200 million individual sequence reads corresponding to 90 million individual molecules. From the 90 million molecules, about 4 million molecules were determined to be significant binders to a protein target. To increase the number of negative examples, one million molecules were added to the 4 million molecules determined to be significant binders to the protein target. The one million negative examples including molecules that that were sequenced but bound only a control not containing the target (non-target control) or to other targets, as well as another million molecules that were not sequenced but were present in the initial libraries. The first data set was then split into training datasets and testing datasets using a Murcko scaffold split Landrum.

To enrich this dataset with intermediate products, all the possible two-building block and three-building block molecules corresponding to a given DNA sequence were determined. A theoretical proportion of each intermediate product was then computed from the estimated building block yields.

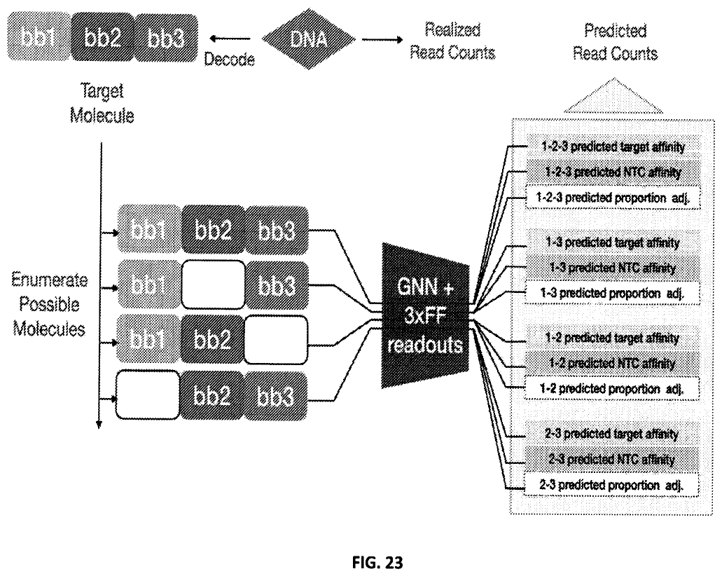

To model the read counts for each DNA tag as following a negative binomial distribution whose mean is driven by multiple factors. For a given DNA tag/read count pair, both full and possible intermediate products are considered factors driving the total read count as illustrated in FIG. 23. shows an overview of the model utilized to process trisynthon as full products and the disynthons as intermediate products to analyze a data set and predict the target biding affinity, non-target binding affinity and a proportion adjustment for each of the full an intermediate products. The full ML model's ability to predict for both the full and intermediate products of a compound allow for the model to learn and provide more insight regarding a compound's binding affinity to a target.

The read count of a DNA tag i in either the NTC or target selection experiment is modeled by a negative binomial distribution with a mean parameter μᵢ and a dispersion parameter a:

C_{target,i}˜NB(μ_{target,i}, α_{target}) and C_{NTC,i}˜NB(μ_{NTC,i}, α_{NTC}). The α values are obtained through a negative binomial regression before training.

For the μᵢ values the binding enrichment of the molecules corresponding to tag i against a given target (B_{target,i}) is due to a combination of the enrichment of the corresponding trisynthon (R_{tri,i}) and three possible disynthons (R_{di}_{₁}_{,i}, R_{di}_{₂}_{,i}, R_{di}_{ⱼ}_{,i}):

$${B}_{target , i} = {p}_{tri , i} {R}_{tri , i} + {\sum }_{j = 1}^{3} {p}_{{di}_{j} , i} {R}_{{di}_{j} , i} \qquad ( 1 )$$

where the p values correspond to the proportions of the trisynthons and disynthons that are present in the final mixture. Similarly, we assume that enrichment of the molecules in the NTC (B_{NTC,i}) is also due to a combination of the enrichment in the trisynthon and disynthons:

$${B}_{NTC , i} = {p}_{tri , i} {R}_{NTC , tri , i} + {\sum }_{j = 1}^{3} {p}_{{di}_{j} , i} {R}_{NTC , {di}_{j} , i} \qquad ( 2 )$$

It was assumed that the counts for a given DNA tag against the target are due to a combination of the binding enrichment to the target, the binding enrichment in the NTC experiment, the starting material estimated using the DLS experiment (C_{dls,i}), and promiscuous binding counts (C_{promiscuity,i}).

Thus, the mean parameter of the negative binomial distribution was set as

$${µ}_{i , target} = σ ( {B}_{target , i} + {β}_{NTC} \cdot {B}_{NTC , i} + {β}_{DLS} {p}_{{dls}_{i}} + {β}_{promiscuity} {c}_{promiscuity} + {β}_{constant} ) \qquad ( 3 )$$

Here, the β values are learned by negative binomial regression on the training set and σ is the softplus function. Similarly, the counts for a given DNA tag against the NTC are explained by the binding enrichment to the NTC, the DLS counts (C_{dls,i}), and promiscuous binding counts (C_{promiscuity,i}). Hence,

$${µ}_{i , NTC} = σ ( {B}_{NTC , i} + {β}_{DLS}^{′} {p}_{dls , i} + {β}_{promiscuity}^{′} {C}_{promiscuity} + {β}_{constant}^{′} ) \qquad ( 4 )$$

Two different ways of estimating the proportion of product k, pₖ. The first is to set these values based on our internal reaction yield dataset: p_{k,lab}. To refine this approach, and take into account the possible noise in the yield measurements, we propose to adjust the estimated proportion with a learned parameter for each molecule: p_{k,adjust}. In this case, pₖ=σ(p_{k,adjust}+p_{k,lab}).

To learn the enrichment values, R_{target,molecule} and R_{NTC,molecule}, two models were used. The input to both being a molecular graph corresponding to a compound. The Weave atom featurizer was employed to encode the atoms and a canonical bond featurizer was used to encode the bonds between atoms. For the first model, we used a single message passing graph neural network followed by a sequence to sequence layer to outputs a 128 dimensional encoding. The 128 dimensional encoding was then transformed using two distinct fully connected networks to get R_{target,i} and R_{NTC,i}. The second model followed the same method as the first model except that the 128 dimensional encoding was also transformed into p_{k,adjust} through an additional fully connected network. These models were then tuned on the training set using batch sizes of 32 and an Adam optimizer with a learning rate of 10⁻³ for 15 epochs.

For a given DNA barcode example i, the negative binomial loss for the target and NTC

$$values were {L}_{value , i} = - log ( P ( {c}_{value , i} \vert {µ}_{value , i} , {α}_{value} ) ) \qquad ( 5 )$$

where the P is the probability mass function of the negative binomial distribution parameterized by μ_{value,i} and αᵥₐₗᵤₑ. The full loss for example i is

$${L}_{i} = {L}_{target , i} + {L}_{ntc , i} + γ \cdot ( {R}_{target , i}^{2} + {R}_{NTC , i}^{2} ) \qquad ( 6 )$$

The model uses the R_{target,I} values during the validation phase to rank and/or score the binding affinities of the compounds to the target.

The ML model capable of processing the full product proportion data, as disclosed herein, was then evaluated against three different models. For the first model the only source of binding enrichment was trisynthons as a source of binding enrichment data. (B_{target,i}=p_{tri,i}R_{tri,i}, B_{NTC,i}=p_{tri,i}R_{NTC,tri,i}). In the second model, the only source of binding enrichment is the disynthons. (B_{target,i}=Σⱼ₌₁³p_{di}_{ⱼ}_{,i}R_{di}_{ⱼ}_{,i},B_{NTC,i}=Σⱼ₌₁³p_{di}_{ⱼ}_{,i},R_{NTC,di}_{ⱼ}_{,i}).

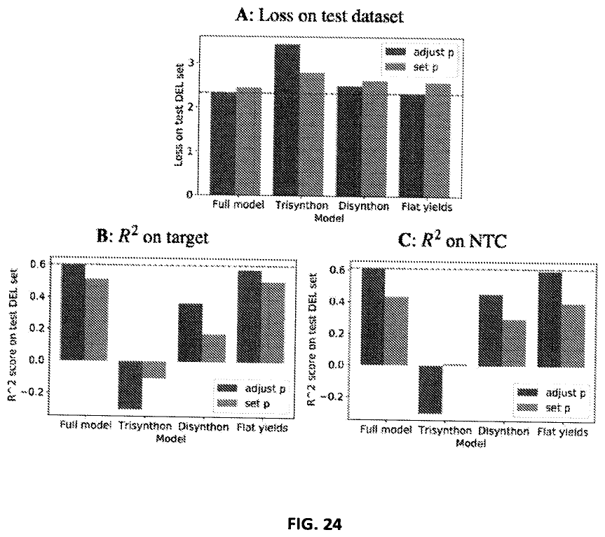

For the third model, it was assumed that each reaction produces a 70% yield, so all p_{di}_{ⱼ}_{,i}=0.147 and all p_{tri,i}=0.147. For each of the models, the ML model described herein and the three evaluate against the ML model an experiment was run where the product proportions are fixed and an additional experiment where an adjustment to the proportions is learned as described above. FIG. 24 illustrates the Loss and R² scores on the test data set. Graph A shows the negative binomial loss on the test data; graph B shows the R² score between the target protein counts and the generated μ_{target,i} counts; and graph C shows the R² score between the NTC counts and the generated μ_{NTC,i} counts.

One evaluation metric was performance on the testing set which is a subset of the internal DEL data. When evaluating the ML model against testing data from the internal DEL data set, the loss was calculated and the R² scores of between the μ_{ntc} and μ_{target} values and the true counts were examined.

Across the two approaches to modelling the product proportions, the trisynthon-only model produces noticeably worse results. This is likely evidence that the trisynthons data by itself is too noisy to produce reliable estimates and does not capture all of the DEL products. The disynthon-only model also produced worse results than the full ML model. The tests showed that modeling disynthons is not only a de-noising step, but is also an integral portion of the data. The model utilizing constant yield data also performed worse, than modeling the yield and product proportion values to train and help model the data. The model with the learned adjustments to yields shows improved performance, likely due to noise in the laboratory processes where several rounds of purification are performed.

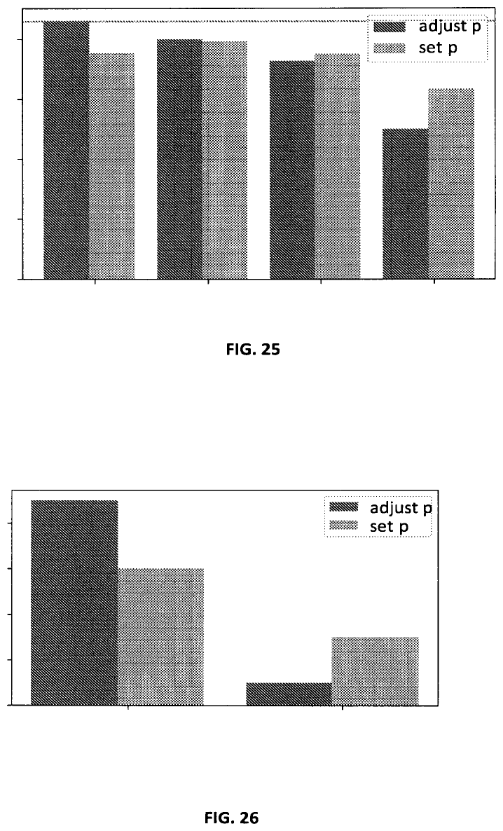

To estimate the generalization ability of the learned model, the performance of the full ML model and the models that it was evaluated against were tested on a dataset of 150 molecules from commercial vendors. The binding affinities were measured internally, and the molecules were classified as binders or non-binders to the target. The enrichment values R_{target,molecule} are used for predicting the binding affinity. The area under the receiver operating characteristic (ROC) curve for various models is shown in FIG. 25. On this dataset, the full model with learned yields outperforms the remaining models.

To further investigate the effects of incorporating yield data, the full ML model and the model with flat yields were tested on the 150 external molecules. The hit rate percent among the top 10 identified hits were examined as illustrated in FIG. 26. These results demonstrate that the full ML model with lab yields and a learned adjustment performed best in a virtual screening application and that setting learned yields makes more of an impact in pertinent individual cases.

Overall, the testing showed that the full ML model described herein outperforms models that do not consider all full and intermediate products and/or a models that do not include proportions of the products, as illustrated in FIGS. 24-26. Using only full product (trisynthon) data produce datasets that were shown to be too noisy to support effective training, and may also inadequately describing the underlying data. Using only intermediate (disynthon) data may be an effective approach to aggregating and de-noising DEL data, but disregards potentially useful data required to effectively screen compounds.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

What is claimed is:

1. A method comprising:
a) a computer implemented method comprising:
i. receiving a first data set comprising: (i) a first compound descriptor for each compound of a first library of compounds, and (ii) a compound fitness score for each compound of the first library of compounds;
ii. training a prediction model on the first data set;
iii. inputting into the model a second data set comprising a second compound descriptor for each compound of a second library of compounds; and
iv. generating from the prediction model a compound fitness score for each compound of the second library of compounds utilizing at least one or more compound descriptors of the first library of compounds and/or one or more compound descriptors of the second library of compounds, and
b) selecting a third library of compounds according to information comprising one or more compound fitness scores of the second library of compounds and/or one or more compound fitness scores of the first library of compounds.

2. The method of claim 1, wherein the third library of compounds comprises: (i) a compound from the second library of compounds, (ii) a compound from the first library of compounds, (iii) a compound comprising two or more compounds from the second library of compounds, (iv) a compound comprising two or more compounds from the first library of compounds, (v) a compound comprising a compound from the second library of compounds and a compound from the first library of compounds, (vi) a compound not present in the first library of compounds or the second library of compounds, (vii) a compound comprising a compound from the second library of compounds and a compound not present in the first library of compounds or the second library of compounds, (viii) a compound comprising a compound from the second library of compounds and a compound not present in the first library or compounds or the second library of compounds, or (ix) a combination of two or more of (i) to (viii).

3. The method of claim 1 or claim 2, wherein the first library a first DNA-encoded library (DEL) and/or the second library is a second DNA-encoded library.

4. The method of any one of claims 1-3, wherein step (b) is part of the computer implemented method.

5. The method of any one of claims 1-3, wherein step (b) is not part of the computer implemented method.

6. The method of any one of claims 1-3, wherein step (b) comprises a first sub-step that is part of the computer implemented method and a second sub-step that is not part of the computer implemented method, wherein the first step and the second step are performed sequentially, and the first sub-step is performed first or the first sub-step is performed second.

7. The method of any one of claims 1-6, wherein the information further comprises an assessment score (sometimes referred to as an external fitness score) of a compound of the second library of compounds and/or an assessment score (sometimes referred to as an external fitness score) of a compound of the first library of compounds.

8. The method of claim 7, wherein the assessment score of the compound of the second library of compounds is a second fitness score generated independently from the compound fitness score generated from the computer implemented method.

9. The method of claim 7 or claim 8, wherein the assessment score of the compound of the first library of compounds is a first fitness score that is different from the compound fitness score for the compound of the first library of compounds.

10. The method of any one of claims 1-9, wherein one or more compounds of the first library is a first test compound (sometimes referred to as a full product or full product compound), a building block(s) of the first test compound, a first byproduct generated during synthesis of the first test compound (sometimes referred to as a byproduct or side product), or an intermediate generated during synthesis of the first test compound (sometimes referred to as an intermediate), or a combination of two or more thereof.

11. The method of claim 10, wherein the first test compound is a desired product of a synthesis reaction comprising two or more of the building blocks of the first test compound.

12. The method of claim 11, wherein the first byproduct is an undesired product of the synthesis reaction comprising the two or more building blocks of the first test compound.

13. The method of any one of claims 1-12, wherein one or more compounds of the second library (or optionally subsequent library as applicable in an iterative method) is a second test compound (sometimes referred to as a full product or full product compound), a building block(s) of the second test compound, a second byproduct generated during synthesis of the second test compound (sometimes referred to as a byproduct or side product), or an intermediate generated during synthesis of the second test compound (sometimes referred to as an intermediate), or a combination of two or more thereof.

14. The method of claim 13, wherein the second test compound is a desired product of a synthesis reaction comprising two or more of the building blocks of the second test compound.

15. The method of claim 14, wherein the second byproduct is an undesired product of the synthesis reaction comprising the two or more building blocks of the second test compound.

16. The method of any one of claims 1-15, wherein one or more compounds of the third library is a third test compound (sometimes referred to as a full product or full product compound), a building block(s) of the third test compound, a third byproduct generated during synthesis of the third test compound (sometimes referred to as a byproduct or side product), or an intermediate generated during synthesis of the third test compound (sometimes referred to as an intermediate), or a combination of two or more thereof.

17. The method of claim 16, wherein the third test compound is a desired product of a synthesis reaction comprising two or more of the building blocks of the third test compound.

18. The method of claim 17, wherein the third byproduct is an undesired product of the synthesis reaction comprising the two or more building blocks of the third test compound.

19. The method of any one of claims 1-18, wherein the first compound descriptor comprises data or information associated with the compound of the first library of compounds, wherein the data or information comprises binding affinity of the compound to a target molecule (optionally wherein the target molecule is a drug target such as a protein or a nucleic acid), activity of the compound, a physical property of the compound (e.g., lipophilicity), toxicity of the compound, stability of the compound, permeability of the compound, sequencing reads associated with an abundance of the compound in an experiment, compound structure, information related to synthesis of the compound, labeling data, process quality control data, yield associated with synthesis of the compound, sequencing data, labeling data, product data, synthesis efficiency data, mass spectrometry data, compound fraction data, binding data, matrix binding data, promiscuity data, structure data, or building block validation data, or a combination of two or more thereof.

20. The method of any one of claims 1-19, wherein the second compound descriptor comprises data or information associated with the compound of the second library of compounds, wherein the data or information comprises binding affinity of the compound to a target molecule (optionally wherein the target molecule is a drug target such as a protein or a nucleic acid), activity of the compound, a physical property of the compound (e.g., lipophilicity), toxicity of the compound, stability of the compound, permeability of the compound, sequencing reads associated with an abundance of the compound in an experiment, compound structure, information related to synthesis of the compound, labeling data, process quality control data, yield associated with synthesis of the compound, sequencing data, labeling data, product data, synthesis efficiency data, mass spectrometry data, compound fraction data, binding data, matrix binding data, promiscuity data, structure data, or building block validation data, or a combination of two or more thereof.

21. The method of claim 19 or claim 20, wherein the compound is a full product compound, intermediate product compound, or byproduct compound.

22. The method of any one of claims 1-21, comprising testing one or more of the compounds of the first library of compounds in an in vitro or in vivo assay.

23. The method of any one of claims 1-22, comprising testing one or more of the compounds of the third library of compounds in an in vitro or in vivo assay.

24. The method of any one of claims 1-23, wherein each compound of the third library of compounds comprises or is synthesized to comprise a nucleic acid tag, the method further comprising sequencing the third library of compounds to generate sequencing data associated with the third library of compounds.

25. The method of any one of claims 1-24, wherein the information of step (b) comprises sequencing data associated with an external library of compounds (e.g., a library comprising nucleic acid tags from each compound in the first library and/or second library).

26. The method of any one of claims 1-25, wherein the compound fitness score for each compound in the first library of compounds is generated from data comprising sequencing data associated with the first library of compounds.

27. The method of claim 24, 25, or 26, wherein the sequencing data comprises a read count, a quality score associated with the read count, and/or comprises a score calculated from the sequencing read count or set of read counts from different experimental conditions from the first library of compounds and/or the second library of compounds.

28. The method of claim 27, wherein the score comprises the read count or the read counts divided by the total number of reads in a selection of compounds or the average number of reads in a selection of compounds, or similar a mathematical function that has utilized a read count (directly or indirectly).

29. The method of any one of claims 1-28, wherein at least one compound fitness score for each compound of the first library of compounds is generated from data comprising a first compound descriptor (e.g., sequencing data, labeling data, product data, synthesis efficiency data, mass spectrometry data, compound fraction data, binding data, matrix binding data, promiscuity data, structure data, or building block validation data, or a combination of two or more thereof).

30. The method of any one of claims 1-29, wherein the prediction model utilizes a probabilistic framework to process the first data set and the second data set, and to output the compound fitness score for each compound of the second library of compounds.

31. The method of any one of claims 10-30, wherein the fitness score is generated at least in part from data from a full product compound comprising a non-target count, a target count, and/or a product proportion adjustment value.

32. The method of any one of claims 10-31, wherein the fitness score is generated at least in part from data from an intermediate product compound comprising a no target control count, a target count, and/or a product proportion adjustment value.

33. The method of any one of claims 1-32, comprising generating a compound fitness score for each compound in the third library of compounds utilizing sequencing data associated with sequencing the third library of compounds.

34. The method of any one of claims 1-33, comprising assaying the third library of compounds.

35. The method of claim 34, wherein the assay comprises binding the third library of compounds to a target.

36. The method of claim 34 or claim 35, wherein the assay comprises sequencing the third library of compounds or a subset of the third library of compounds (e.g., wherein the subset is a subset of compounds that binds to the target).

37. The method of any one of claims 1-36, wherein the fitness score of any one of the compounds comprises a binding and/or activity score of the compound.

38. The method of any one of claims 1-37, wherein the third library comprises one or more compounds from the second library with a compound fitness score greater than a threshold score.

39. The method of any one of claims 1-39, wherein the method comprises pre-processing the first data set and/or the second data set.

40. The method of claim 39, wherein the pre-processing step is performed before step i and/or before step iii of the computer implemented method.

41. The method of any one of claims 1-40, comprising refining a fitness score generated from the prediction model, optionally wherein the refinement is performed by the prediction model, and/or optionally wherein refining comprises incorporating information from an external library (e.g., a library of nucleic acid tags associated with the first library and/or second library of compounds).

42. The method of any one of claim 1-41, wherein the second library comprises one or more different compounds than the first library.

43. The method of any one of claim 1-42, wherein the second library comprises one or more compounds different from the first library.

44. The method of any one of claim 1-45, further comprising repeating steps ii-vi to update the model.

45. The method of any one of claim 1-44, wherein the full product comprises a trisynthon and the intermediate product comprises a disynthon and/or monosynthon.

46. The method of claims 1-45, wherein steps (a)-(b) are iteratively repeated to identify a set of potential compounds with one or more desired properties.

47. The method of any one of claims 1-46, wherein steps (a)-(b) are iteratively repeated to identify a set of potential compounds with one or more desired compound fitness scores.

48. The method of any one of claim 1-47, wherein a compound fitness score is in relation to an oral drug solubility, intestinal absorption, a permeability, a hERG toxicity, a CYP inhibition, a blood brain barrier permeability, a P-glycoprotein activity, plasma protein binding, and/or a binding affinity of any one of the compounds.

49. The method of any one of claim 1-48, wherein the first compound descriptor input into the model comprises compound structure and/or experimental data.

50. The method of any one of claim 1-49, wherein the prediction model is a machine learning model.

51. The method of claim 50, wherein the machine learning model comprises a neural network.

52. The method of claim 51, wherein the neural network is a graph neural network.

53. The method of any one of claims 50-52, wherein the machine learning model comprises a graph neural network and an attention layer.

54. The method of claim 53, wherein the neural network is a graph attention network.

55. The method of any one of claim 1-54, wherein the method comprises performing a validation assay on at least one compound of the third library of compounds.

56. The method of any one of claim 1-55, wherein the method comprises performing low-throughput analysis on at least one compound of the third library of compounds.

57. The method of any one of claim 1-56, wherein the method comprises inputting a third data set comprising a compound descriptor and a compound fitness score for each compound of the third library of compounds into a secondary system in a validation assay.

58. The method claim 57, further comprising inputting data from the validation assay into the prediction model.

59. The method of claim 57 or claim 58, wherein the validation assay comprises a proxy for binding or biochemical activity including one or more of absorbance, fluorescence, luminescence, radioactivity, NMR, crystallography, microscopy including cryo-electron microscopy, mass spectrometry, or Raman scattering, for example, Surface Plasmon Resonance (SPR) measures the reflection of polarized light, which can detect the change in the reflection angle (refractive index), and immobilization or binding of a ligand (compound) to the surface (which contains the immobilized target protein) affects the mass or thickness of the surface, which changes the refraction.

60. The method of any one of claim 1-59, wherein the prediction model generates a predictive compound descriptor for each compound in the first library of compound and/or the second library of compounds.

61. The method of claim 60, wherein the compound fitness score is generated at least in part from the predictive compound descriptor for each of the compounds.

62. The method of any one of claims 1-61, wherein the first library of compounds is about 10,000 compounds to about hundred billion compounds or about 10,000 compounds to about ten billion compounds; and wherein the second library of compounds is about 10,000 compounds to about hundred billion compounds or about 10,000 compounds to about ten billion compounds.

63. A computer-implemented system comprising: a digital processing device comprising: at least one processor, an operating system configured to perform executable instructions, a memory, and a computer program including instructions executable by the digital processing device to perform the method of any one of claims 1-62.

64. A non-transitory computer-readable storage media encoded with a computer program including instructions executable by one or more processors to perform the method of any one of claims 1-62.
